(19) United States
(12) Patent Application Publication
(10) Pub. No.: US 2006/0040385 A1
(43) Pub. Date: Feb. 23, 2006

(54) Cultured human pancreatic islets, and uses thereof

(75) Inventors: Joseph Itskovitz-Eldor, Haifa (IL); Hanna Segev, Hogla (IL); Bettina Fishman, Haifa (IL)

Correspondence Address: Martin Moynihan;Anthony Castorina, Suite 207, 2001 Jefferson Davis Highway, Arlington, VA, 22202, US

(73) Assignee: Technion Research & Development Foundation Ltd., Haifa (IL)

(21) Appl. No.: 10/536,734

(22) PCT Filed: Dec. 3, 2003
(86) PCT No.: PCT/IL03/01027

§ 371 Date: May 27, 2005

Related U.S. Application Data
(60) Provisional application No. 60/430,935, filed on Dec. 5, 2002.
(60) Provisional application No. 60/446,516, filed on Feb. 12, 2003.

(51) Int. Cl. C12N 5/08; A61K 35/39

(52) U.S. Cl. 435/366; 424/93.7

(57) ABSTRACT

A method of generating cells capable of secreting insulin is disclosed. The method comprises subjecting mammalian embryonic stem cells to set of culturing conditions suitable for differentiation of at least a portion thereof into cells displaying at least one characteristic associated with a pancreatic islet cell progenitor phenotype, and subjecting such differentiated cells to a set of culturing conditions suitable for formation of surface bound cell clusters including insulin producing cells.

### FIELD AND BACKGROUND OF THE INVENTION

The present invention relates to methods of generating mammalian insulin secreting cells and tissues by in-vitro culture, and to uses of such cells and tissues for treating diseases associated with insulin deficiency. More particularly, the present invention relates to methods of generating optimally functional human pancreatic beta cells and pancreatic islets by in-vitro culture of human embryonic stem cells, and to methods of using such cells and islets to treat diabetes mellitus in humans.

Diabetes mellitus is a devastating, life-long disease associated characterized by high mortality and morbidity (reviewed in The Diabetes Control and Complication Trial Research Group, 1993. N Engl J Med. 329:977-986). This disease causes long-term complications which may affect virtually all parts of the body. In particular, diabetes frequently results in retinopathy leading to blindness, cardiovascular disease, stroke, nephropathy leading to kidney failure, and neuropathy (nerve damage), and may require amputation of affected body parts. Furthermore, diabetes may lead to complications during pregnancy, such as birth defects in babies born to women with the disease. If not diagnosed and treated with insulin, a person with diabetes may lapse into a life-threatening diabetic coma, also known as diabetic ketoacidosis.

World-wide, diabetes occurs in nearly 5 percent of the population ranging in age from 20 to 79 years, and hence affects 150 million people. In the United States alone, an estimated 17 million people-over 6 percent of the population-have diabetes mellitus, and each year about 1 million Americans aged 20 or older are diagnosed with the disease. In 1999, about 450,000 deaths occurred among adults with diabetes in the United States. Ominously, the prevalence of diabetes in the United States is increasing due to the increasing frequency among the population of segments at increased risk, such as the elderly, the obese, Hispanic Americans and other minority groups. According to recent estimates, the frequency of diabetes in the United States is expected to reach nearly nine percent of the population by 2025. Beyond its direct health impact, diabetes, due to its widespread prevalence and severity, is also a disease of great economic impact, as highlighted by the fact that for the year 1997 alone, the economic cost of diabetes in the United States was estimated at about one hundred billion dollars.

Diabetes is a disease caused by uncontrolled blood glucose levels as a result of a malfunction in the capacity of the body to metabolize glucose, its main source of metabolic energy, due to either lack or defect of insulin, a hormone secreted by the pancreas which uniquely functions to lower blood glucose levels. The pancreas is an organ both structurally and functionally compartmentalized into two discrete endocrine and exocrine components. The function of the endocrine pancreas is to produce blood glucose controlling hormones such as insulin, as well as glucagon, somatostatin, and pancreatic polypeptide, whereas the function of the exocrine pancreas is production of digestive enzymes, such as amylase and lipase, and sodium bicarbonate. The endocrine cells of the pancreas are organized in approximately one million highly vascularized and innervated micro-organs, termed islets of Langerhans (pancreatic islets), which are distributed throughout the pancreas, and which represent approximately 1 percent of the total mass of the pancreas. The pancreatic islets, which resume pancreatic endocrine physiology, include the four islet cell types; glucagon producing alpha cells, insulin producing beta cells, somatostatin producing delta cells, and pancreatic polypeptide producing PP cells. The most abundant cells of the pancreatic islets are the beta cells. Beta cells form clusters connected by gap junctions which respond synchronously when glucose reaches stimulatory concentrations of 7 to 10 millimolar (Martin and Soria, 1996. Cell Calcium 20:409-414; Nadal et al., 1999. J Physiol. (Lond.) 517:85-93; Charollais et al., 2000. J Clin Invest. 106:235-243; and Soria et al., 2000. Pflügers Archiv-European J Physiol. 440:1-18.). In contrast, the glucagon secreting alpha cells, which are inactive at higher glucose concentrations, and hence active when beta cells are silent, do not display synchronous activation (Nadal et al., 1999. J Physiol. (Lond.) 517:85-93; Quesada et al., 1999. of Langerhans. Diabetes 48:2390-2397). The somatostatin producing delta cells, which have a paracrine function within the islet are also unsynchronized (Soria et al., 2000. Pflügers Archiv-European J Physiol. 440:1-18). The pulsatile insulin release of the islets of Langerhans is due to their integrated physiology (Santos et al., 1991. Pflügers Arch.-Eur J Physiol. 418:417-422; Martin et al., 1995. Diabetes 44:300-305; Martin and Soria, 1995. J Physiol. (Lond.) 486:361-371), and alterations in this physiological property are observed early during development of diabetes.

Diabetes is broadly classified into two major types: the insulin dependent type (type 1 diabetes; juvenile diabetes) and the non insulin dependent type (type 2 diabetes; insulin resistant diabetes).

Type 1 diabetes is an autoimmune disease affecting approximately 5 million people worldwide, and which accounts for 5 to 10 percent of diagnosed diabetes in the United States. The pathogenesis of type 1 diabetes involves autoimmune attack and gradual elimination of the insulin secreting beta cells of the pancreas. The consequence is a gradual shut-down of the insulin secreting capacity of the pancreas, and, as a result, sufferers of type 1 diabetes typically require daily insulin injections to survive. Presently, the causes of the body's immune rejection of its own beta cells remain unknown, however genetic factors, and environmental factors, such as viruses, are thought to be involved. Type 1 diabetes occurs most often in young adults and children, however the condition can occur at any age. Symptoms of type 1 diabetes usually appear over a short period, however actual beta cell depletion may begin years earlier.

Type 2 diabetes is the most common form of diabetes, accounting for about 90 to 95 percent of cases. This type of diabetes usually develops in adults aged 40 and older and, however its highest incidence occurs in adults over the age of fifty-five. Type 2 diabetes is often associated with a metabolic syndrome which includes obesity, elevated blood pressure, and high blood lipid levels. Approximately 80 percent of people suffering from type 2 diabetes are classified as obese. Furthermore, the incidence type 2 diabetes is increasing among children and adolescents in the United States due to increasing obesity rate in this population.

At the time of diagnosis of type 2 diabetes the pancreatic insulin producing capacity is typically adequate. However, due to a condition of unknown etiology, termed insulin resistance, the body does not utilize the insulin effectively. The course of the disease typically involves decrease of insulin production after a period of several years, which ultimately leads to the same devastating consequences as type 1 diabetes.

There is therefore a critical need for novel and optimal methods of treating diabetes mellitus.

One strategy which has been attempted for treating diabetes involves transplanting donor derived beta cells or pancreatic islets to a recipient suffering from the disease (reviewed in Soria et al., 2001. Differentiation 68:205-219). Such a treatment modality is potentially highly potent since it can endow recipients with a capacity for physiologically regulated insulin responses capable of curing type 1 and some cases of type 2 diabetes. However, this procedure is highly restricted due the difficulty of obtaining two pancreases from suitably haplotype matched adult donors from which to isolate the 700,000 to 900,000 islets required for effective transplantation (Shapiro et al., 2000. N Eng J Med. 343:230-238).

One strategy which has been proposed to overcome the difficulty in obtaining sufficient numbers of pancreatic beta cells or pancreatic islets involves generating sufficient numbers of such cells or tissues by in-vitro culture.

Several prior art approaches have been attempted for generating in-vitro cultures of insulin secreting cells or tissues being suitable for treating diabetes.

One approach involves culturing of cells derived from adult or fetal human pancreatic tissues in attempts to generate cultures of insulin producing pancreatic cells (Zhao M. et al., 2002. Transplantation 73:1454-1460; Bonner-Weir S. et al., 2000. Proc Natl Acad Sci U S A. 97:7999-8004; and Itkin-Ansari P. et al., 2000. Molecular Endocrinology 14:814-822).

Another approach involves genetically transforming human cell lines with the homeodomain transcription factor PDX-1 in attempts to generate cultures of beta cells (Itkin-Ansari P. et al., 2000. Molecular Endocrinology 14:814-822).

Yet another approach involves genetically transforming mouse embryonic stem cell cultures with the homeodomain transcription factor PDX-1 via retroviral transduction in attempts to generate cultures of insulin secreting cells (Itkin-Ansari P. et al., 2000. Molecular Endocrinology 14:814-822).

Still another approach involves employing a cell trapping system using an insulin promoter regulated selectable marker gene to isolate insulin secreting cell clones from cultured mouse undifferentiated embryonic stem cells (Soria et al., 2000. Diabetes 49:1-6).

A further approach involves administering insulin secreting cells derived from cultured mouse embryonic stem cells (Soria et al., 2000. Diabetes 49:1-6) or cultured cadaveric human donor derived pancreatic islets (Zhao M. et al., 2002. Transplantation 73: 1454-1460) to treat streptozotocin induced diabetes in mice.

Yet a further approach involves culturing of mouse embryoid bodies under conditions for inducing differentiation of pancreatic progenitor cells followed by conditions for inducing proliferation of such progenitors followed by conditions for inducing formation of surface bound cell clusters in attempts to generate islet like cell clusters containing insulin expressing cells (Lumelsky et al., 2001. Science 292:1389-1394).

Still a further approach involves culturing of human embryonic stem cells under non adherent conditions on uncoated plastic in attempts to generate embryoid bodies containing insulin secreting cells (Schuldiner M. et al., 2000. Proc Natl Acad Sci U S A. 97:11307-11312; Assady S. et al., 2001. Diabetes 50:1691-1697).

An additional approach involves culturing of human embryonic stem cells under adherent conditions on coated plastic in attempts to generate insulin secreting cells (Assady S. et al., 2001. Diabetes 50:1691-1697).

However, all of the aforementioned approaches suffer from significant disadvantages.

Prior art approaches utilizing culturing of animal cells in attempts to generate insulin secreting cells are suboptimal for modeling cell culture methods applicable to human cells, and are unsatisfactory for generating cells suitable for human administration.

Prior art approaches aimed at generating adherent cells or tissues are suboptimal due to such cells or tissues requiring cumbersome, harmful, and/or inefficient manipulations for harvesting.

Prior art approaches employing culture of adult or fetal donor derived cells or tissues in attempts to generate cultures of insulin secreting cells or tissues have the disadvantages of relying on donor derived materials which are prohibitively difficult to obtain, unsuitable for generating pancreatic cells at desired developmental stages, and have the limited proliferative potential of differentiated cells.

Prior art approaches using culture of immortalized cell lines in attempts to generate cultures of insulin secreting cells have the disadvantages of generating such cells inefficiently, of generating cells not having the potential of embryonic stem cells to generate pancreatic cells at early developmental stages, of having karyotypic and/or phenotypic abnormalities, and hence being unsuitable for treatment of pancreatic disease in humans.

Prior art approaches involving generation of genetically transformed insulin expressing cells have the disadvantages of requiring genetic manipulation which is cumbersome and inefficient, and/or of generating cells being inherently unacceptably risky and unproven for human administration as a consequence of their being genetically transformed.

Prior art approaches using administration of cultured insulin secreting cells or tissues in attempts to treat streptozotocin induced diabetes in mice employ an artificially induced animal disease model which does not satisfactorily model human pancreatic disease, and/or involve administration of insulin secreting cells from animals.

Prior art approaches involving culturing mouse embryonic stem cells in attempts to generate cell clusters containing insulin expressing cells have the disadvantages of generating cultures of islet like clusters containing a suboptimal proportion of insulin secreting cells, of displaying an unsatisfactory insulin secretion capacity, of having an unsatisfactorily high content of non clustered cells, and/or of exhibiting suboptimal in-vitro longevity.

Prior art approaches involving culturing of human embryonic stem cells under non adherent conditions in attempts to generate embryoid bodies containing insulin secreting cells have the disadvantages of producing cultures containing a suboptimal maximal proportion of insulin secreting cells, of containing cells having an unsatisfactory maximal insulin secretion capacity, and of not having the capacity to generate islet like cell clusters.

Thus, all prior art approaches have failed to provide an adequate solution for generating in-vitro cultures of insulin secreting cells or tissues being suitable for treating diabetes in humans.

There is thus a widely recognized need for, and it would be highly advantageous to have, a method of generating in-vitro cultures of insulin secreting cells or tissues devoid of the above limitation.

### SUMMARY OF THE INVENTION

According to one aspect of the present invention there is provided a method of treating a pancreatic disease in a subject, the method comprising: (a) subjecting mammalian embryonic stem cells to a first set of culturing conditions selected suitable for differentiation of at least a portion of the mammalian embryonic stem cells into cells displaying at least one characteristic associated with a pancreatic islet cell progenitor phenotype; (b) subjecting the cells displaying at least one characteristic associated with a pancreatic islet cell progenitor phenotype to a second set of culturing conditions selected suitable for formation of surface bound cell clusters including insulin producing cells; and (c) administering a therapeutically effective dose of the insulin producing cells to the subject, thereby treating the pancreatic disease.

According to further features in preferred embodiments of the invention described below, the method of treating a pancreatic disease further comprises isolating the surface bound cell clusters and optionally the insulin producing cells therefrom prior to step (c).

According to still further features in preferred embodiments, the method of treating a pancreatic disease further comprises: (i) dissociating the surface bound cell clusters into single cells including the insulin producing cells; and (ii) subjecting the single cells to a third set of culturing conditions selected suitable for maintaining the insulin producing cells in culture for at least 14 days prior to step (c).

According to still further features in preferred embodiments, the method of treating a pancreatic disease further comprises isolating the insulin producing cells prior to step (c).

According to still further features in preferred embodiments, the method of treating a pancreatic disease further comprises isolating the suspended cell clusters prior to step (c).

According to still further features in preferred embodiments, the method of treating a pancreatic disease further comprises the step of selectively harvesting the mammalian embryonic stem cells from a culture including feeder cells and the mammalian embryonic stem cells prior to step (a).

According to still further features in preferred embodiments, the method of treating a pancreatic disease further comprises isolating the cells displaying at least one characteristic associated with a pancreatic islet cell progenitor phenotype prior to step (b).

According to still further features in preferred embodiments, the method of treating a pancreatic disease further comprises (d) dissociating the cells displaying at least one characteristic associated with a pancreatic islet phenotype into single cells displaying at least one characteristic associated with a pancreatic islet phenotype; and (e) subjecting the single cells displaying at least one characteristic associated with a pancreatic islet cell progenitor phenotype to a fifth set of culturing conditions selected suitable for proliferation of the cells displaying at least one characteristic associated with a pancreatic islet cell progenitor phenotype prior to step (b).

According to still further features in preferred embodiments, the insulin producing cells are syngeneic with or allogeneic with the subject.

According to still further features in preferred embodiments, the subject is a human or a non human mammal.

According to still further features in preferred embodiments, step (c) is effected by administering the isolated surface bound cell clusters to the subject.

According to still further features in preferred embodiments, step (c) is effected by administering the suspended cell clusters to the subject.

According to still further features in preferred embodiments, step (c) is effected by administering the isolated suspended cell clusters to the subject.

According to still further features in preferred embodiments, the administering is effected by transplantation or injection of the insulin producing cells into the pancreas of the subject.

According to another aspect of the present invention there is provided a method of producing insulin, the method comprising: (a) subjecting mammalian embryonic stem cells to a first set of culturing conditions selected suitable for differentiation of at least a portion of the mammalian embryonic stem cells into cells displaying at least one characteristic associated with a pancreatic islet cell progenitor phenotype; and (b) subjecting the cells displaying at least one characteristic associated with a pancreatic islet cell progenitor phenotype to a second set of culturing conditions selected suitable for formation of surface bound cell clusters including insulin producing cells, thereby producing the insulin.

According to further features in preferred embodiments of the invention described below, the method of producing insulin further comprises: (c) harvesting the insulin.

According to still further features in preferred embodiments, the method of producing insulin further comprises: (c) isolating the surface bound cell clusters and optionally isolating the insulin producing cells therefrom.

According to still further features in preferred embodiments, the method of producing insulin further comprises: (c) dissociating the surface bound cell clusters into single cells including the insulin producing cells; and (d) subjecting the single cells to a third set of culturing conditions selected suitable for maintaining the insulin producing cells in culture for at least 14 days.

According to still further features in preferred embodiments, the method of producing insulin further comprises: (e) isolating the insulin producing cells.

According to still further features in preferred embodiments, the method of producing insulin further comprises: (e) isolating the suspended cell clusters.

According to still further features in preferred embodiments, the method of producing insulin further comprises the step of selectively harvesting the mammalian embryonic stem cells from a culture including feeder cells and the mammalian embryonic stem cells prior to step (a).

According to still further features in preferred embodiments, the method of producing insulin further comprises isolating the cells displaying at least one characteristic associated with a pancreatic islet cell progenitor phenotype prior to step (b).

According to still further features in preferred embodiments, the method of producing insulin further comprises (c) dissociating the cells displaying at least one characteristic associated with a pancreatic islet phenotype into single cells displaying at least one characteristic associated with a pancreatic islet phenotype; and (d) subjecting the single cells displaying at least one characteristic associated with a pancreatic islet cell progenitor phenotype to a fifth set of culturing conditions selected suitable for proliferation of the cells displaying at least one characteristic associated with a pancreatic islet cell progenitor phenotype prior to step (b).

According to still further features in preferred embodiments, the second set of culturing conditions includes culturing the cells displaying at least one characteristic associated with a pancreatic islet cell progenitor phenotype in a culturing medium, and wherein harvesting the insulin is effected by harvesting the culture medium.

According to yet another aspect of the present invention there is provided a method of generating cells capable of secreting insulin, the method comprising: (a) subjecting mammalian embryonic stem cells to a first set of culturing conditions selected suitable for differentiation of at least a portion of the mammalian embryonic stem cells into cells displaying at least one characteristic associated with a pancreatic islet cell progenitor phenotype; and (b) subjecting the cells displaying at least one characteristic associated with a pancreatic islet cell progenitor phenotype to a second set of culturing conditions selected suitable for formation of surface bound cell clusters including insulin producing cells, thereby generating cells capable of secreting insulin.

According to further features in preferred embodiments of the invention described below, the method of generating cells capable of secreting insulin further comprises: (c) isolating the surface bound cell clusters and optionally isolating the insulin producing cells therefrom.

According to still further features in preferred embodiments, the method of generating cells capable of secreting insulin further comprises: (c) dissociating the surface bound cell clusters into single cells including the insulin producing cells; and (d) subjecting the single cells to a third set of culturing conditions selected suitable for maintaining the insulin producing cells in culture for at least 14 days.

According to still further features in preferred embodiments, the method of generating cells capable of secreting insulin further comprises: (e) isolating the insulin producing cells.

According to still further features in preferred embodiments, the third set of culturing conditions is selected suitable for maintaining the insulin producing cells in suspended cell clusters.

According to still further features in preferred embodiments, the suspended cell clusters are characterized by a proportion of the insulin producing cells of at least 4 percent.

According to still further features in preferred embodiments, an insulin secretion rate capacity of the insulin producing cells of the suspended cell clusters is at least 6 microunits insulin per one hundred thousand cells per hour.

According to still further features in preferred embodiments, a total insulin secretion capacity of the insulin producing cells of the suspended cell clusters is at least 0.50 microunits insulin per one hundred thousand cells.

According to still further features in preferred embodiments, the method of generating cells capable of secreting insulin further comprises: (e) isolating the suspended cell clusters.

According to still further features in preferred embodiments, the third set of culturing conditions is selected suitable for inhibiting growth of substantially non insulin producing cells.

According to still further features in preferred embodiments, the substantially non insulin producing cells are neurons and/or mesenchymal cells.

According to still further features in preferred embodiments, the dissociating the surface bound cell clusters into single cells is effected by trypsinization of the surface bound cell clusters.

According to still further features in preferred embodiments, the third set of culturing conditions includes a condition selected from the group consisting of a substantially serum free culture medium, a basic fibroblast growth factor free culture medium, a culture medium including nicotinamide, a culture medium including a synthetic serum supplement, a culture medium including glucose at a concentration of 15 millimolar or less, and inhibiting adherence of the insulin producing cells to a surface.

According to still further features in preferred embodiments, inhibiting adherence of the insulin producing cells to the surface is effected by culturing the insulin producing cells on a substantially non cell adherent plastic surface.

According to still further features in preferred embodiments, the method of generating cells capable of secreting insulin further comprises the step of selectively harvesting the mammalian embryonic stem cells from a culture including feeder cells and the mammalian embryonic stem cells prior to step (a).

According to still further features in preferred embodiments, the first set of culturing conditions is selected suitable for inducing formation of embryoid bodies.

According to still further features in preferred embodiments, the first set of culturing conditions is selected capable of inhibiting adherence of the mammalian embryonic stem cells to a surface.

According to still further features in preferred embodiments, inhibiting adherence of the mammalian embryonic stem cells to a surface is effected by culturing the mammalian embryonic stem cells on a substantially non cell adherent plastic surface.

According to still further features in preferred embodiments, the at least one characteristic associated with a pancreatic islet cell progenitor phenotype is expression and optionally display of nestin.

According to still further features in preferred embodiments, the method of generating cells capable of secreting insulin further comprises isolating the cells displaying at least one characteristic associated with a pancreatic islet cell progenitor phenotype prior to step (b).

According to still further features in preferred embodiments, the isolating is effected by subjecting the cells displaying at least one characteristic associated with a pancreatic islet cell progenitor phenotype to a fourth set of culturing conditions selected suitable for inhibiting growth of cells not displaying the at least one characteristic associated with a pancreatic islet cell progenitor phenotype.

According to still further features in preferred embodiments, the method of generating cells capable of secreting insulin further comprises (c) dissociating the cells displaying at least one characteristic associated with a pancreatic islet phenotype into single cells displaying at least one characteristic associated with a pancreatic islet phenotype; and (d) subjecting the single cells displaying at least one characteristic associated with a pancreatic islet cell progenitor phenotype to a fifth set of culturing conditions selected suitable for proliferation of the cells displaying at least one characteristic associated with a pancreatic islet cell progenitor phenotype prior to step (b).

According to still further features in preferred embodiments, the fourth set of culturing conditions includes a culturing condition selected from the group consisting of a substantially serum free culture medium, a culture medium including insulin, a culture medium including transferrin, a culture medium including fibronectin, a culture medium substantially including selenium, and facilitating adherence of the cells displaying at least one characteristic associated with a pancreatic islet cell progenitor phenotype to a surface.

According to still further features in preferred embodiments, the facilitating adherence of the cells displaying at least one characteristic associated with a pancreatic islet cell progenitor phenotype to a surface is effected by culturing the cells displaying at least one characteristic associated with a pancreatic islet cell progenitor phenotype in contact with a tissue culture coated plastic surface.

According to still further features in preferred embodiments, the fifth set of culturing conditions includes a condition selected from the group consisting of a substantially serum free culture medium, a culture medium including basic fibroblast growth factor, a culture medium including a synthetic serum supplement, and facilitating adherence of the cells displaying at least one characteristic associated with a pancreatic islet cell progenitor phenotype to a surface.

According to still further features in preferred embodiments, the facilitating adherence of the cells displaying at least one characteristic associated with a pancreatic islet cell progenitor phenotype to a surface is effected by culturing the cells displaying at least one characteristic associated with a pancreatic islet cell progenitor phenotype in contact with a plastic surface coated with gelatin or poly-L-lysine.

According to still further features in preferred embodiments, the second set of culturing conditions is selected suitable for formation of cell clusters including insulin producing cells capable of displaying a change in an insulin secretion in response to a drug selected from the group consisting of an increase in the insulin secretion wherein the drug is tolbutamide, an increase in the insulin secretion wherein the drug is IBMX, a decrease in the insulin secretion wherein the drug is diazoxide, a decrease in the insulin secretion wherein the drug is nifedipine, and a decrease in the insulin secretion wherein the drug is carbachol.

According to still further features in preferred embodiments, the second set of culturing conditions includes a condition selected from the group consisting of a substantially serum free culture medium, a basic fibroblast growth factor free culture medium, a culture medium including nicotinamide, a culture medium including a synthetic serum supplement, a culture medium including glucose at a concentration of 15 millimolar or less, and facilitating adherence of the cells displaying at least one characteristic associated with a pancreatic islet cell progenitor phenotype to a surface.

According to still further features in preferred embodiments, the facilitating adherence of the cells displaying at least one characteristic associated with a pancreatic islet cell progenitor phenotype to a surface is effected by culturing the cells displaying at least one characteristic associated with a pancreatic islet cell progenitor phenotype in contact with a plastic surface coated with gelatin.

According to still further features in preferred embodiments, the second set of culturing conditions is selected suitable for formation of cell clusters including insulin producing cells maintainable in culture for at least 7 days.

According to still further features in preferred embodiments, the third set of culturing conditions is selected suitable for formation of cell clusters including cells displaying at least one characteristic associated with a pancreatic islet cell phenotype selected from the group consisting of an endocrine cell precursor phenotype, an alpha cell phenotype, a beta cell phenotype, a delta cell phenotype, and a neuronal cell phenotype.

According to still further features in preferred embodiments, the at least one characteristic associated with an endocrine cell precursor phenotype is expression or display of an mRNA of a transcription factor or an mRNA of a glucose transporter.

According to still further features in preferred embodiments, the transcription factor is Pax6.

According to still further features in preferred embodiments, the glucose transporter is Glut2.

According to still further features in preferred embodiments, the at least one characteristic associated with an alpha cell phenotype is expression or display of glucagon mRNA or glucagon.

According to still further features in preferred embodiments, the at least one characteristic associated with a beta cell phenotype is selected from the group consisting of expression or display of an mRNA of a transcription factor, an mRNA of a glucose transporter, an mRNA of a glucose metabolism enzyme, and insulin mRNA.

According to still further features in preferred embodiments, the transcription factor is selected from the group consisting of Pdx1, Isl1, Beta2, Pax4 and Nkx6.1.

According to still further features in preferred embodiments, the glucose transporter is Glut2.

According to still further features in preferred embodiments, the glucose metabolism enzyme is glucokinase.

According to still further features in preferred embodiments, the at least one characteristic associated with a delta cell phenotype is expression or display of somatostatin.

According to still further features in preferred embodiments, the at least one characteristic associated with a neuronal cell phenotype is a neuronal morphology.

According to still further features in preferred embodiments, the mammalian embryonic stem cells are human embryonic stem cells.

According to still further features in preferred embodiments, the human embryonic stem cells are selected from the group consisting of I6 cells, H9 cell derived cells, and H13 cells.

According to still another aspect of the present invention there is provided an insulin producing cell cluster comprising insulin producing cells being maintainable in culture for at least 14 days, wherein a proportion of the insulin producing cells in the cell cluster is at least 4 percent.

According to further features in preferred embodiments of the invention described below, the proportion of the insulin producing cells in the cell cluster is at least 32 percent.

According to still further features in preferred embodiments of the invention described below, the insulin secretion rate capacity of the insulin producing cells is at least 6 microunits insulin per one hundred thousand cells per hour.

According to still further features in preferred embodiments of the invention described below, a total insulin secretion capacity of the insulin producing cells is at least 0.50 microunits insulin per one hundred thousand cells.

According to still further features in preferred embodiments, the cell cluster further comprises cells displaying at least one characteristic associated with a pancreatic islet cell phenotype selected from the group consisting of an endocrine cell precursor phenotype, an alpha cell phenotype, a beta cell phenotype, a delta cell phenotype, and a neuronal cell phenotype

According to still further features in preferred embodiments, the insulin producing cell cluster produces human insulin.

According to still further features in preferred embodiments, the insulin producing cell cluster includes human cells.

According to still further features in preferred embodiments, the human cells have a genotype of I6 cells, H9 cell derived cells, and H13 cells.

According to still further features in preferred embodiments, the H9 cell derived cells are H9.2 cells.

The present invention successfully addresses the shortcomings of the presently known configurations by providing a method of generating cultured human embryonic stem cell derived pancreatic islet like cell clusters being more optimally differentiated, having a higher proportion of insulin producing cells, comprising cells having a higher insulin secretion rate capacity and a higher total insulin secretion capacity, displaying greater in-vitro longevity, and being more suitable for treating human diabetes than all prior art cultured embryonic stem cell derived islet like clusters. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific-reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a flow chart depicting the culturing protocol employed to generate the insulin secreting beta cells and islet like clusters of the present invention from human embryonic stem cells.

FIGS. 1b-g are photomicrographs depicting the appearance of the cultured human embryonic stem cells of the present invention during Stages I to VI of culture, respectively.

FIG. 2 is a histogram depicting a comparison of insulin secretion levels in undifferentiated embryonic stem cells (Stage I) and Stage VI suspended islet like clusters.

FIG. 3 is a set of photographs depicting agarose gel electrophoretic analysis of RT-PCR amplified pancreas specific gene expression in cultured cells/cell clusters.

FIG. 4 is a histogram depicting markedly higher levels of insulin secretion in Stage V surface bound islet like clusters grown in Medium 3 (5 millimolar glucose) compared to such clusters grown in modified Medium 3 (17.5 millimolar; the glucose concentration of Medium 2 used for Stage IV culturing conditions).

FIGS. 5a-c are photomicrographs depicting high proportions of insulin, glucagon, and somatostatin expressing cells, respectively, in Stage V surface bound islet like clusters, following immunohistochemical staining.

FIG. 6 is a histogram depicting beta cell specific insulin secretion profiles by Stage V surface bound islet like clusters in response to treatment with 10 micromolar tolbutamide, 100 micromolar IBMX, 50 micromolar nifedipine, 500 micromolar diazoxide, and 10 micromolar carbachol. Assays were performed under “low glucose” (3.3 millimolar) or “high glucose” (16.7 millimolar) conditions.

FIG. 7 is a histogram depicting a very sharp increase in insulin secretion levels in Stage VI suspended islet like clusters relative to Stage V surface bound islet like clusters.

FIGS. 8a-d are immunofluorescent confocal photomicrograph sections depicting expression of insulin in 80 percent of cells in Stage VI suspended islet like clusters immunofluorescently stained for insulin. Stage VI clusters were plated on gelatin coated 13 mm glass cover slides and stained with guinea pig anti-insulin primary antibody and FITC conjugated anti-rabbit IgG secondary antibody. The slides were analyzed using a confocal microscope (Bio-Rad MRC 1024, Richmond, Calif.).

FIGS. 9a-i are a confocal microscopy photomicrographs depicting immuno-staining for insulin, C-peptide, somatostatin and glucagon of stage VI clusters. FIGS. 9a, 9d and 9g respectively depict C-peptide staining, somatostatin staining and glucagon staining (all in green). FIGS. 9b, 9d and 9h respectively depict insulin staining (red). FIGS. 9c, 9f and 9i respectively depict co-staining of insulin and C-peptide, somatostatin and glucagon. Co-staining is seen as orange. Slides were analyzed using a confocal microscope. Bar=10 microns.

FIG. 10 is a fluorescent photomicrograph depicting TUNEL+ and insulin stained stage VI clusters. Stage VI cells were stained for TUNEL+ nuclei (green) using In Situ Cell Death Detection Kit followed by immunofluoresence staining with mouse anti-insulin antibody (red). Slides were analyzed using a confocal microscope. Bar=10 microns.

### DESCRIPTION OF THE PREFERRED EMBODIMENTS

The present invention is of methods of generating mammalian pancreatic cells and tissues from stem cells and of methods of using such cells and tissues to produce pancreatic hormones and to treat pancreatic disease in mammals. Specifically, the present invention can be used to generate cultured insulin secreting beta cell like cells and pancreatic islet like cell clusters including such cells by in-vitro culture of human embryonic stem cells. As such, the present invention can be used to produce human insulin and to treat diabetes in humans.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Treatment of diabetes by daily injection of insulin, the standard prior art therapy, does not satisfactorily prevent the debilitating or lethal consequences of this disease.

Treatment of pancreatic diseases by transplantation of pancreatic islets to recipients suffering from such diseases is technically feasible, however this strategy can not be routinely practiced due to the extreme difficulty of obtaining sufficient numbers of haplotype matched islets from adult donors.

Various approaches of using in-vitro culture to generate insulin producing cells or tissues suitable for treating diabetes have been attempted in the prior art. One approach involves culturing of cells derived from donor derived differentiated cells or tissues in attempts to generate insulin producing cells or tissues. Another approach involves genetically transforming differentiated cells in the context of various strategies in attempts to generate insulin producing cells. Still another approach involves culturing of embryonic stem cells in attempts to generate embryoid bodies containing insulin producing cells. Yet another approach involves culturing of embryonic stem cells in attempts to generate adherent cultures of insulin producing cells. An additional approach involves modeling treatment of diabetes by administering cultured insulin producing cells or tissues to animals with chemically induced diabetes.

However, all of the aforementioned prior art approaches have significant disadvantages.

Approaches employing culture of donor derived differentiated cells or tissues in attempts to generate insulin producing cells or tissues are suboptimal since these utilize cells or tissues which are difficult to obtain, are unsuitable for generating pancreatic cells at desired developmental stages, have the restricted proliferative potential of differentiated cells, or require the use of karyotypically and phenotypically abnormal immortalized cell lines. Strategies involving generation of genetically transformed insulin producing cells are unsatisfactory since these require genetic manipulation of cells which is cumbersome and inefficient, and since the use of genetically modified cells is of unproven safety and efficacy for human administration. Approaches involving culturing of embryonic stem cells in attempts to generate embryoid bodies containing insulin producing cells are suboptimal since such embryoid bodies contain suboptimal proportions of insulin producing cells, have an unsatisfactory insulin secretion capacity, and since such approaches can not be used to generate pancreatic islet like tissues. Strategies involving culturing of embryonic stem cells in attempts to generate adherent cultures of insulin producing cells are unsatisfactory since such adherent cultures contain suboptimal proportions of insulin producing cells, have an unsatisfactory insulin secretion capacity, exhibit suboptimal in-vitro longevity and/or do not produce pancreatic islet like tissues. Approaches involving modeling treatment of diabetes by administering cultured insulin producing cells or tissues to animals with chemically induced diabetes are suboptimal since these employ an artificially induced animal disease model which does not satisfactorily model human diabetes. Indeed, all approaches employing animal cells are unsatisfactory for accurately modeling culturing of human materials and for producing cells or tissues suitable for human administration.

Thus, all prior art approaches employing in-vitro culturing of cells and tissues have failed to provide cultures having an optimal proportion of insulin producing cells, having an optimal insulin production capacity, having optimal in-vitro longevity, comprising pancreatic islet like tissues, and being optimal for human administration.

While experimenting with cultured human embryonic stem cells, the present inventors unexpectedly uncovered that culturing human embryonic stem cells according to the method of the present invention could be used to generate, for the first time, purified islet like cell clusters which are highly differentiated and functional, and are characterized by a proportion of insulin producing cells corresponding to the upper physiological range, and being far greater than that of all prior art cultured islet like clusters. It was further uncovered that such culturing could also be used to generate cultures of islet like cell clusters having a significantly higher insulin production capacity and in-vitro longevity than all prior art embryonic stem cell derived cultures of islet like cell clusters.

Hence, in sharp contrast to prior art cultured embryonic stem cell derived cells and tissues, the cultured islet like cell clusters including the cells of the present invention can be generated in essentially unlimited numbers and thereby used to treat pancreatic diseases such as diabetes in humans, to identify drugs or treatments having desired effects on human islet and islet cell physiology, to optimize application of such treatments, to test the toxicity of compounds on human pancreatic islets and islet cells, to model the pathogenesis of human diabetes, and to characterize human islet and islet cell biology including aspects such as gene expression, development, and physiology.

Method of Generating Insulin Secreting Cells

Thus, according to the present invention there is provided a method of generating cells capable of secreting insulin.

The method is effected by first subjecting mammalian stem cells to culturing conditions suitable for differentiation of at least a portion thereof into cells displaying at least one characteristic associated with a pancreatic islet cell progenitor phenotype (also referred to herein as “islet cell progenitors”).

In a second culturing step of the method of the present invention, the islet cell progenitors are subjected to culturing conditions suitable for formation of surface bound cell clusters which include insulin producing cells being preferably maintainable in culture for at least 7 days.

Preferably, the insulin producing cells of the present invention are capable of displaying: (i) an increase in insulin secretion in response to tolbutamide; (ii) an increase in insulin production in response to IBMX; (iii) a decrease in insulin secretion in response to diazoxide; (iv) a decrease in insulin secretion in response to nifedipine; and/or (v) a decrease in insulin secretion in response to carbachol.

As used herein, the phrase “islet cell progenitor” refers to a partially differentiated cell capable of differentiating into one of the islet cell types.

As used herein, the term “islet”, refers to an islet of Langerhans.

According to a preferred embodiment of the method of the present invention, the islet cell progenitors are preferably isolated prior to the second culturing step described above (formation of surface bound clusters). Such isolation is advantageous for generating surface bound clusters including an enriched proportion of the insulin producing cells of the present invention. As described in the Examples section below, such enrichment is associated with a decreased proportion of mesenchymal cells which serves to increase the longevity of the insulin producing cells of the present invention in culture by preventing overgrowth of mesenchymal cells.

Isolation of the islet cell progenitors prior to the second culturing step can be advantageously effected by subjecting the islet cell progenitors to culturing conditions suitable for inhibiting growth of non islet cell progenitor cells.

Prior to the second culturing step, the islet cell progenitors are preferably dissociated into single cells and subjected to culturing conditions suitable for proliferation thereof.

Dissociating the islet cell progenitors and subjecting them to culturing conditions suitable for their proliferation can be used for generating optimal numbers of such cells which, in turn, can be used for generating optimal numbers of surface bound clusters, and/or surface bound clusters including an optimal proportion of the insulin producing cells of the present invention.

According to the present invention, the islet cell progenitors are preferably subjected to the culturing conditions suitable for inhibiting growth of non islet cell progenitor cells prior to being dissociated and subjected to the culturing conditions suitable for their proliferation. This enables optimal proliferation of the islet cell progenitors, since depletion of non islet cell progenitor cells provides culture space and nutrients for proliferation of the islet cell progenitors which would otherwise be used by highly proliferative non islet cell progenitors cells such as mesenchymal cells.

Preferably, following formation thereof, the surface bound clusters are dissociated into single cells, including the insulin producing cells of the present invention. The dissociated cells are subjected to culturing conditions primarily suitable for maintaining the cells in culture for at least 14 days.

These culturing conditions are preferably also selected suitable for: (i) maintaining the insulin producing cells of the present invention in suspended clusters whose proportion of such cells is at least 4 percent; (ii) generating cells having an insulin secretion capacity of least 6 microunits insulin per one hundred thousand cells per hour; (iii) generating cells having a total insulin secretion capacity of at least 0.50 microunits insulin per one hundred thousand cells; (iv) inhibiting the growth of substantially non insulin producing cells; and/or (v) formation of suspended cell clusters including cells displaying at least one characteristic associated with a pancreatic islet cell phenotype such as an islet endocrine cell precursor phenotype, an islet alpha cell phenotype, an islet beta cell phenotype, an islet delta cell phenotype, and/or an islet neuronal cell phenotype.

In order to enable generation of cultures which include an optimal proportion of insuring producing cells of the present invention and which exhibit a longevity of at least 14 days, the dissociated cells described above are preferably isolated prior to culturing.

Thus, the present invention provides methodology which is suitable for the generation of insulin producing cells or clusters. As is illustrated in the Examples section which follows, such cells or clusters are characterized by optimal pancreatic beta cell specific and pancreatic islet specific differentiation and function, respectively. Critically such cells have the capacity to secrete optimally high levels of insulin, and may be optimally purified and expanded to essentially unlimited numbers in culture.

The following section describes embodiments of the present methodology in more detail; additional description can also be found in the Examples section which follows.

Stem Cells

While stem cells from essentially any mammalian species may be cultured according to the above described method to generate the insulin producing cells of the present invention, the method is preferably effected using human stem cells. Use of human cells is highly advantageous for generating insulin producing cells of the present invention and clusters including such cells being optimally suitable for human specific applications relative to prior art cultured embryonic stem cell derived insulin producing cell clusters and cells which have only been generated using mouse cells (for example, refer to Lumelsky et al., 2001. Science 292:1389-1394). Such human specific applications include, for example, producing human insulin, or treating pancreatic disease in humans.

Various types of human embryonic stem cells may be employed to generate the insulin producing cells of the present invention and islet like clusters including such cells.

Preferably, cells of an established embryonic stem cell line cultured according to standard art methodology on a layer of mitotically inactivated mouse embryonic feeder (MEF) cells are used. Such feeder cells serve to maintain the stem cells in an undifferentiated state in such cultures. Alternately, primary stem cells from the inner cell mass of a blastocyst, or embryonic stem cells generated by de-differentiation of differentiated cells may be employed.

Alternately, embryonic stem cells derived from differentiated cells may be employed. Ample guidance for obtaining stem cells from differentiated cells, such as adult cells, is provided in the literature of the art (for example, refer to: Wilmut, I. et al., 1997. Nature 385:810-813.).

While cells of various types of human cell lines are suitable for practicing the method of the present invention, such cells are preferably cells of the I6 cell line, cells of an H9 derived cell line, or cells of the H113 cell line. Preferably, the H9 derived cell line is cell line H9.2.

Ample guidance for obtaining, culturing and manipulating stem cell lines such as the above described cell lines is provided in the literature of the art (for example, refer to: Thomson, J. A. et al., 1998. Science 282:1145-1147; and Reubinoff B E. et al., 2000. Nat Biotech. 18:399-404).

Since, as mentioned hereinabove, feeder cells function to inhibit differentiation of stem cells, stem cells from stem cell lines cultured on a feeder cell layer are preferably substantially isolated from such feeder cells prior to being subjected to the culturing conditions suitable for generating the islet cell progenitors.

Culturing Conditions

Islet Cell Progenitor Generation

As described hereinabove, the method of generating the insulin producing cells of the present invention is effected by subjecting the stem cells to culturing conditions suitable for differentiation of at least a portion thereof into cells displaying at least one characteristic associated with a pancreatic islet cell progenitor phenotype. Preferably, this characteristic is expression and/or display of nestin.

Nestin is a cytoskeletal protein widely recognized in the art as being a characteristic marker of islet cell progenitors (for example, refer to: Hunziker E. and Stein M., 2000. Biochem Biophys Res Commun. 271:116-9; Zulewski H. et al., 2001. Diabetes 50:521-33; Soria B., 2001. Differentiation 68:205-19).

Expression/display of nestin protein may be conveniently monitored via standard immunohistochemical techniques by using anti nestin antibodies to stain fixed cells (for example, refer to Lumelsky et al., 2001. Science 292:1389-1394).

Preferably, the islet cell progenitors are generated by subjecting the stem cells to culturing conditions suitable for inducing formation of embryoid bodies, and/or including inhibiting adherence of the stem cells to a surface.

Embryoid bodies are easily recognized by the ordinarily skilled artisan as being coalesced embryonic stem cells displaying a characteristic structure and morphology in culture (for example, refer to Schuldiner M. et al., 2000. Proc Natl Acad Sci U S A. 97:11307-11312) which include nestin positive islet cell progenitor cells (for example, refer to: Shamblott M J., 2001. Proc. Natl. Acad. Sci. USA. 98: 113-118; Lumelsky et al., 2001. Science 292:1389-1394).

Embryoid bodies may be optimally generated from stem cells by culturing such cells under conditions including inhibiting adherence thereof to a surface.

Various methods of inhibiting adherence cultured stem cells to a surface so as to facilitate generation of embryoid bodies therefrom are known to the ordinarily skilled artisan.

Preferably, according to the method of the present invention, the embryoid bodies are generated by culturing the stem cells on a substantially non cell adherent plastic surface. This may be conveniently achieved, for example, by culturing the stem cells in a plastic Petri dish. Ample guidance for generating embryoid bodies by culture of stem cells in plastic Petri dishes is provided in the literature of the art (for example, refer to: Assady S. et al., 2001. Diabetes 50:1691-1697; Itskovitz-Eldor J. et al., 2000. Molecular Medicine 6:88-95; Soria et al., 2000. Diabetes 49:1-6; and Schuldiner M. et al., 2000. Proc Natl Acad Sci U S A. 97:11307-11312). Inhibiting adherence of embryoid bodies cultured over a surface can be enhanced by culturing such embryoid bodies with shaking. Alternately, the embryoid bodies may be generated using the hanging drop technique, a method in which cells are cultured in a drop of medium adhering to the underside of an elevated surface (for example, refer to: Wobus, A M. et al., 1991. Differentiation 48:173).

As described and illustrated in the Examples section below, culturing isolated stem cells in a plastic Petri dish under the culturing conditions described therein can be used to efficiently generate embryoid bodies. Essentially the same culturing conditions have been used in the prior art to achieve essentially the same results (for example, refer to: Schuldiner M. et al., 2000. Proc Natl Acad Sci U S A. 97:11307-11312; Lumelsky et al., 2001. Science 292:1389-1394).

Isolation of Islet Cell Progenitors

As described hereinabove, the islet cell progenitors are preferably isolated prior to being subjected to the culturing conditions suitable for formation of the surface bound clusters.

According to the method of the present invention, the islet cell progenitors are optimally isolated by subjecting embryoid bodies containing such cells to culturing conditions suitable for inhibiting growth of non islet cell progenitor cells.

Such isolation is preferably effected using culturing conditions including: (i) a culture medium being substantially serum free; (ii) a culture medium supplemented with insulin, transferrin, fibronectin, and/or selenium; and/or (iii) facilitating adherence of the islet cell progenitors to a surface.

Depending on the purpose and application, culturing conditions including various combinations of the above described medium compositions and facilitating cell adherence to a surface may be suitable for inhibiting the growth of cells other than the islet cell progenitors.

Preferably, inhibiting the growth of non islet cell progenitor cells is achieved using culture conditions including a serum free culture medium containing all of the aforementioned supplements and facilitating adherence of the islet cell progenitors to a surface.

Various methods may be employed for facilitating adherence of the islet cell progenitors to a surface under such culturing conditions.

According to the present invention, facilitating such adherence under such conditions is optimally effected by culturing the embryoid bodies under static conditions in a tissue culture flask having a cell contacting surface specially treated for facilitating cell adherence. Such flasks are routinely used in the art for adherent cell culture and are commercially available from various sources (for example, from Nunc). Alternately, a plastic Petri dish coated with a protein promoting cell adhesion, such as gelatin or poly-L-lysine, may be employed, as described hereinbelow.

As is described and illustrated in the Examples section below, culturing the embryoid bodies in a tissue culture flask in a serum free culture medium supplemented with insulin, transferrin, fibronectin, and selenium, according to the culturing protocol set forth therein, can be used to efficiently inhibit growth of non islet cell progenitor cells. Similar or essentially identical culturing conditions have been used to achieve similar or essentially identical results in the prior art using mouse cells (for example, refer to: Lee S. H., et al., 2000. Nature Biotechnol. 18:675; Lumelsky et al., 2001. Science 292:1389-1394).

Expansion of Isolated Islet Cell Progenitors

As previously described, the islet cell progenitors are preferably dissociated into single cells and cultured under conditions suitable for proliferation thereof prior to being subjected to the culturing conditions suitable for formation of surface bound clusters.

Various methods of generating single islet cell progenitors may be employed. Preferably, this is effected by treating the embryoid bodies with EDTA, as described and illustrated in the Examples section below. Alternately, this may be achieved by treating the embryoid bodies with a proteolytic enzyme such as type IV collagenase, dispase, or trypsin, optionally in combination with EDTA (for example, as described in: Itskovitz-Eldor J et al., 2000. Mol Med 6:88-95; Schuldiner M. et al., 2000. Proc Natl Acad Sci U S A. 97:11307-11312; and Soria et al., 2000. Diabetes 49:1-6).

The culturing conditions suitable for proliferation of the islet cell progenitors preferably include: (i) a substantially serum free culture medium; (ii) a culture medium supplemented with basic fibroblast growth factor (bFGF) and/or a synthetic serum supplement; and/or (iii) facilitating adherence of the islet cell progenitors to a surface.

Depending on the purpose and application, culturing conditions including various combinations of the above described medium compositions and facilitating adherence of such cells to a surface may be suitable for proliferation of the islet cell progenitors.

Preferably, the culturing conditions suitable for proliferation of the islet cell progenitors include a serum free culture medium containing all of the aforementioned supplements and facilitating adherence of such cells to a surface.

Preferably the synthetic serum supplement is a supplement designed for selective differentiation of neuronal progenitors. Without being bound to a paradigm, the present inventors are of the opinion that the observations that such a supplement facilitating differentiation of neuronal cells also facilitates differentiation of islet cells due to both cell types being derived from a common nestin positive progenitor.

Preferably, the synthetic serum supplement is B27 serum supplement or N2 serum supplement (both from Invitrogen), more preferably a combination of both.

Various methods may be employed for facilitating adherence of the islet cell progenitors to a surface under such culturing conditions. Preferably, facilitating such adherence under such conditions is effected by culturing such cells in contact with a plastic surface coated with gelatin or poly-L-lysine. Alternately, this may be effected by culturing such cells in a tissue culture flask treated to facilitate cell adherence, for example as described hereinabove.

According to the present invention, culturing such cells in contact with a plastic surface coated with gelatin is optimally performed by culturing the embryoid bodies under essentially static conditions in a plastic Petri dish coated with gelatin or poly-L-lysine.

As is described and illustrated in the Examples section below, culturing the embryoid bodies in a plastic Petri dish coated with gelatin or poly-L-lysine, using a serum free culture medium supplemented with bFGF, and B27 and N2 serum supplements, according to the culturing protocol set forth therein, can be used to efficiently induce proliferation of the islet cell progenitors. Similar or essentially identical culturing conditions have been used to achieve similar or essentially identical results in the prior art using mouse cells (for example, refer to: Lee S. H., et al., 2000. Nature Biotechnol. 18:675; Lumelsky et al., 2001. Science 292:1389-1394).

Surface Bound Cluster Formation

As described hereinabove, following differentiation thereof, the islet cell progenitors are subjected to culturing conditions being suitable for formation of the surface bound cell clusters, and being preferably further suitable for formation of cell clusters including insulin producing cells of the present invention capable of displaying: (i) an increase in insulin secretion in response to tolbutamide; (ii) an increase in insulin in response to IBMX; (iii) a decrease in insulin secretion in response to diazoxide; (iv) a decrease in insulin secretion in response to nifedipine; and/or (v) a decrease in insulin secretion in response to carbachol.

According to the present invention, the set of culturing conditions suitable for formation of surface bound cell clusters preferably includes: (i) a culture medium substantially free of serum and/or bFGF; (ii) a culture medium including nicotinamide, a synthetic serum supplement, and/or glucose at a concentration of 15 millimolar or less; and/or (iii) facilitating adherence of the islet cell progenitors to a surface.

Depending on the purpose and application, culturing conditions including various combinations of the above described medium compositions and facilitating adherence of such cells to a surface may be suitable for formation of the surface bound clusters.

Preferably, the culturing conditions suitable for formation of the surface bound clusters include a serum free and bFGF free culture medium containing all of the aforementioned supplements and facilitating adherence of such cells to a surface.

Preferably the synthetic serum supplement is a supplement designed for selective differentiation of neuronal progenitors.

Preferably, the synthetic serum supplement is B27 serum supplement or N2 serum supplement (both from Invitrogen), more preferably a combination of both.

Preferably, the glucose concentration is selected from the range of 1 to 15 millimolar, more preferably is selected from the range of 2 to 10 millimolar, more preferably is selected from the range of 3 to 7 millimolar, more preferably is selected from the range of 4 to 6 millimolar, and most preferably is about 5 millimolar.

As used herein the term “about” refers to ±10 percent.

Various methods may be employed for facilitating adherence of the islet cell progenitors to a surface under the culturing conditions suitable for formation of the surface bound clusters.

Preferably, facilitating such adherence under such culturing conditions is effected by culturing such cells in contact with a plastic surface coated with gelatin. Alternately, this may be effected by culturing such cells in contact with a plastic surface coated with poly-L-lysine or by culturing such cells in a tissue culture flask treated to facilitate cell adherence, for example as described hereinabove.

According to the present invention, culturing such cells in contact with a plastic surface coated with gelatin is optimally performed by culturing the embryoid bodies under static conditions in a plastic Petri dish coated with gelatin.

Prior to employing the insulin producing cells of the present invention in applications such as treatment of pancreatic disease, as described hereinbelow, it may be desirable to monitor the capacity thereof to display the aforementioned drug responses.

Such monitoring may be conveniently effected in-vitro by culturing such cells in medium including about 3.3 millimolar glucose and treating such cells with the above described drugs at concentrations of about 10 micromolar for tolbutamide, about 100 micromolar for IBMX, about 50 micromolar for nifedipine, about 500 micromolar for diazoxide, and about 10 micromolar for carbachol, and measuring insulin secretion according to the protocol set forth in the Examples section below.

As is described and illustrated in the Examples section below, culturing the embryoid bodies in a plastic Petri dish coated with gelatin, using a serum free and bFGF free culture medium supplemented with B27 and N2 serum supplements, nicotinamide, and 5 millimolar glucose, according to the culturing protocol set forth therein, can be used for formation of surface bound islet like clusters including insulin producing cells of the present invention maintainable in culture for 7 days, and capable of displaying an increase in insulin secretion in response to tolbutamide, an increase in insulin in response to IBMX, a decrease in insulin secretion in response to diazoxide, a decrease in insulin secretion in response to nifedipine, and/or a decrease in insulin secretion in response to carbachol.

With the exception of the carbachol response, similar or essentially identical culturing conditions have been used to achieve similar or essentially identical results in the prior art using mouse cells (for example, refer to: Lee S. H., et al., 2000. Nature Biotechnol. 18:675; Lumelsky et al., 2001. Science 292:1389-1394).

Thus, the insulin producing cells of the present invention display similar changes in insulin secretion in response to tolbutamide, IBMX, diazoxide, and nifedipine as normal pancreatic beta cells [for example, refer to: Trube G. et al., 1998. Pflugers Arch. 407:493-9 (tolbutamide and diazoxide); Montague W. and Cook J R., 1971. Biochem J. 122:115-120 (IBMX); Ahren B. et al., 1990. Prog Brain Res. 84:209-218 (carbachol); and Rojas W. et al., 1990. FEBS Lett. 261:265-270 (nifedipine)]. As such, the insulin producing cells of the present invention optimally recapitulate the insulin production physiology of normal pancreatic beta cells.

The capacity of the insulin producing cells of the present invention to display a non beta cell specific decrease in insulin secretion in response to carbachol, a drug which upregulates insulin secretion in pancreatic beta cells can be used to modulate the balance of insulin production between the insulin producing cells of the present invention and normal beta cells in applied contexts where both cell types are exposed to the same physiological stimuli, as described in further detail hereinbelow.

Suspended Cluster Formation

As described hereinabove, the surface bound clusters are preferably dissociated into single cells, including insulin producing cells of the present invention, and such insulin producing cells are subjected to culturing conditions being suitable for maintaining insulin producing cells in culture for at least 14 days, and being preferably further suitable for: (i) generating suspended cell clusters having a proportion of the insulin producing cells of the present invention of at least 4 percent; (ii) generating insulin producing cells of the present invention having an insulin secretion capacity of least 6 microunits insulin per one hundred thousand cells per hour, and/or having a total insulin secretion capacity of at least 0.50 microunits insulin per one hundred thousand cells; and/or inhibiting the growth of substantially non insulin producing cells; and/or (iii) formation of cell clusters including cells displaying at least one characteristic associated with an islet endocrine cell precursor phenotype, an islet alpha cell phenotype, an islet beta cell phenotype, an islet delta cell phenotype, and/or an islet neuronal cell phenotype.

In order to generate suspended clusters optimally suitable for applications requiring optimal insulin production, such as the pancreatic disease treatment and insulin production methods described hereinbelow, it is preferable for such clusters to include an optimal proportion of the insulin producing cells of the present invention.

Thus, the proportion of the insulin producing cells of the present invention in the suspended cell clusters is preferably at least 4 percent, more preferably at least 10 percent, more preferably at least 15 percent, more preferably at least 20 percent, more preferably at least 25 percent, at least 32 percent, more preferably at least 35 percent, more preferably at least 40 percent, more preferably at least 45 percent, more preferably at least 50 percent, more preferably at least 55 percent, more preferably at least 60 percent, more preferably at least 65 percent, more preferably at least 70 percent, more preferably at least 75 percent, and most preferably at least 80 percent.

As described hereinabove, the culturing conditions suitable for maintaining the insulin producing cells of the present invention in culture for at least 14 days are preferably further suitable for formation of cell clusters including cells displaying at least one characteristic associated with an islet endocrine cell precursor phenotype, an islet alpha cell phenotype, an islet beta cell phenotype, an islet delta cell phenotype, and/or an islet neuronal cell phenotype.

Clusters including cells displaying at least one characteristic associated with various combinations of such islet cell phenotypes may be suitable, depending on the purpose and application.

Preferably, the clusters include cells displaying at least one characteristic associated with the islet beta cell phenotype.

In order to have the capacity to optimally perform pancreatic islet functions the clusters preferably include cells displaying at least one characteristic associated with two such islet cell phenotypes, more preferably three such islet cell phenotypes, and most preferably all of the aforementioned islet cell phenotypes.

Preferably, the characteristic associated with the islet endocrine cell precursor phenotype is expression or display of an mRNA of a transcription factor or glucose transporter.

Preferably, the transcription factor is Pax6 and the glucose transporter is Glut2.

Preferably, the characteristic associated with the islet alpha cell phenotype is expression or display of glucagon mRNA or glucagon.

Preferably, the characteristic associated with the islet beta cell phenotype is expression or display of an mRNA of a transcription factor, a glucose transporter, a glucose metabolism enzyme, or insulin.

Preferably, the transcription factor is Pdx1, Isl1, Beta2, Pax4 or Nkx6.1; the glucose transporter is Glut2; and the glucose metabolism enzyme is glucokinase.

Preferably, the characteristic associated with the islet delta cell phenotype is expression or display of somatostatin, and the characteristic associated with a neuronal cell phenotype is a neuronal morphology.

As described hereinabove, the surface bound clusters may be advantageously dissociated so as to generate single cells, including insulin producing cells of the present invention, prior to subjecting such cells to the culturing conditions suitable for maintaining the insulin producing cells of the present invention in culture for at least 14 days.

Various way of dissociating the surface bound clusters so as to generate single cells including insulin producing cells of the present invention may be employed.

According to the present invention, such dissociation is preferably effected by treatment of the clusters with trypsin, preferably in combination with EDTA, as described and demonstrated in the Examples section which follows. Alternately, such dissociation may be effected by treatment with type IV collagenase or dispase, alone or in combination with about 0.5 molar EDTA, or by treatment with EDTA in the absence of an exogenous proteolytic enzyme. The use of dispase for dissociating islets may be particularly advantageous to generate optimally viable single cells (Josefsen K. et al., 1996. J Endocrinol. 149:145-54). Ample guidance for dissociating pancreatic islets or islet like cell clusters is provided in the literature of the art (for example, refer to Josefsen K. et al., 1996. J Endocrinol. 149:145-54).

As described hereinabove, following surface bound cluster dissociation, the insulin producing cells of the present invention may be advantageously isolated prior to being subjected to the culturing conditions suitable for maintaining the insulin producing cells of the present invention in culture for at least 14 days.

Various ways of isolating the single insulin producing cells of the present invention following dissociation of the surface bound clusters may be employed.

For example, such single cells can be isolated by isolating the surface bound clusters from non clustered cells prior to dissociating the clusters. Such cluster isolation may be effected by mechanical dissection thereof under stereoscopic observation with a pulled glass micropipette or microscalpel, followed by harvesting of the dissected clusters with a pulled glass micropipette by suction. Alternately, the single insulin producing cells of the present invention may be isolated from isolated or non isolated surface bound clusters using standard FACS sorting with a detection antibody specific for a specific surface marker of pancreatic beta cells, such as, for example, glucose transporter-2 or the inward recitifying potassium ion channel Kir-6.2.

According to the present invention, in order to optimally generate the insulin producing cells of the present invention, the set of culturing conditions suitable for maintaining such cells in culture for at least 14 days preferably includes: (i) a culture medium substantially free of serum and/or bFGF; (ii) a culture medium including nicotinamide, a synthetic serum supplement, and/or glucose at a concentration of 15 millimolar or less; and/or (iii) inhibiting adherence of such cells to a surface.

Depending on the purpose and application, culturing conditions including various combinations of the above described medium compositions and inhibiting adherence of such cells to a surface may be suitable.

Preferably, the culturing conditions include a serum free and bFGF free culture medium containing all of the aforementioned supplements and facilitating adherence of such cells to a surface.

Preferably the synthetic serum supplement is designed for selective differentiation of neuronal progenitors.

Preferably, the synthetic serum supplement is B27 serum supplement or N2 serum supplement (both from Invitrogen), more preferably a combination of both.

Preferably, the glucose concentration is selected from the range of 1 to 15 millimolar, more preferably is selected from the range of 2 to 10 millimolar, more preferably is selected from the range of 3 to 7 millimolar, more preferably is selected from the range of 4 to 6 millimolar, and most preferably is about 5 millimolar.

Preferably, the culturing conditions suitable for formation of the surface bound clusters and those suitable for maintaining the insulin producing cells of the present invention in culture for at least 14 days employ substantially identical culturing media.

Various techniques may be employed to inhibit adherence of the single insulin producing cells of the present invention to a surface.

Preferably, inhibiting such adherence is effected by culturing such cells on a substantially non cell adherent plastic surface. Such inhibition of adherence may be enhanced by culturing such cells with shaking. Alternately, such inhibition of adherence may be achieved by culturing such cells using the hanging drop culture technique described hereinabove.

According to the present invention, culturing the cells on a substantially non cell adherent plastic surface is optimally effected by culture thereof in a plastic Petri dish.

Preferably, the non islet cell types whose growth is inhibited by the culturing method suitable for maintaining the insulin producing cells of the present invention in culture for at least 14 days are neurons, more preferably mesenchymal cells.

As is described and illustrated in the Examples section below, dissociating the surface bound clusters into single cells, and culturing such single cells in a plastic Petri dish using a serum free and bFGF free culture medium supplemented with B27 and N2 serum supplements, nicotinamide, and 5 millimolar glucose, according to the culturing protocol set forth therein, can be used to: (i) generate cultures of isolated suspended pancreatic islet like clusters having a proportion of the insulin producing cells of the present invention of 80 percent; (ii) generate clusters being substantially depleted of mesenchymal cells and neurons; (iii) generate clusters including cells displaying characteristics associated with an islet endocrine cell precursor phenotype such as expression or display of a Pax6 mRNA and Glut2 mRNA; (iv) generate clusters including cells displaying characteristics associated with an islet alpha cell phenotype such as expression or display of glucagon mRNA or glucagon; (v) generate clusters including cells displaying characteristics associated with an islet beta cell phenotype such as expression or display of Pdx1 mRNA, Isl1mRNA, Beta2 mRNA, Pax4 mRNA, Nkx6.1 mRNA, Glut2 and glucokinase; (vi) generate clusters including cells displaying characteristics associated with an islet delta cell phenotype such as expression or display of somatostatin; (vii) generate clusters including cells displaying characteristics associated with an islet neuronal cell phenotype such as neuronal morphology; and (viii) generate clusters including insulin producing cells of the present invention being maintainable in culture for at least 14 days, having an insulin secretion capacity of 955 microunits insulin per one hundred thousand cells per hour, and having a total insulin secretion capacity of 955 microunits insulin per one hundred thousand cells.

Advantages of Present Invention Over Prior Art

Insulin Secretion Levels

The maximal proportion of insulin producing cells of prior art cultured islet like clusters is 3.6 percent for suspended/human clusters (Zhao M. et al., 2002. Transplantation 73:1454-1460) or 31.5 percent for surface bound/mouse clusters (Lumelsky et al., 2001. Science 292:1389-1394).

The capacity to generate cultured embryonic stem cell derived islet like clusters whose proportion of insulin producing cells is at least 4 percent is a feature unique to the islet like clusters including the insulin producing cells of the present invention relative to prior art cultured embryonic stem cell derived islet like clusters. The 80 percent proportion of insulin producing cells achievable in the clusters including the insulin producing cells of the present invention represents a 22-fold increase over the maximal proportion of 3.6 percent of insulin producing cells contained in prior art cultured embryonic stem cell derived islet like clusters (Lumelsky et al., 2001. Science 292:1389-1394), and corresponds to the maximal proportion of the insulin producing beta cells in normal pancreatic islets (Soria et al., 2001. Differentiation 68:205-219).

The capacity to maintain the insulin producing cells of the present invention in culture for at least 14 days following dissociation thereof from surface bound cell clusters is another unique feature of the present invention relative to all prior art methods of generating cultured embryonic stem cell derived insulin producing cells or cell clusters. Such a capacity provides optimal flexibility for the timing of use of the insulin producing cells of the present invention.

The ability to maintain the insulin producing cells of the present invention in isolated, suspended cell clusters in cultures essentially depleted of non clustered cells is yet another feature unique to the present invention relative to all prior art cultured embryonic stem cell derived islet like clusters. Such ability enables rapid, efficient, and robust harvesting of purified preparations of the suspended clusters without the need for the harmful enzymatic treatments, such as the trypsinization or collagenase treatments, required for dislodging prior art cultured embryonic stem cell derived surface bound islet like cell clusters (Lumelsky et al., 2001. Science 292:1389-1394). Such ability further enables cluster isolation without the inherent contamination with non clustered/non islet cell types of preparations of such prior art clusters. Such enzymatic treatments result in significant degradation at both the cell and cluster levels, and thereby lead to suboptimal yields of undegraded insulin producing cells and clusters relative to cell and clusters generated according to the method of the present invention. It will be appreciated by the ordinarily skilled artisan that the optimal yields of undegraded cells and clusters including the insulin producing cells of the present invention are optimal for essentially any application.

The ability of the method of the present invention to generate cultured embryonic stem cell derived islet like clusters including insulin producing cells having an insulin secretion rate capacity of least 6 microunits insulin per one hundred thousand cells per hour, and a total insulin secretion capacity of at least 0.50 microunits insulin per one hundred thousand cells are both still further unique features of the present invention relative to prior art methods of generating cultured embryonic stem cell derived islet like clusters.

The maximal insulin secretion of prior art cultured islet like clusters is 2.9 nanograms insulin per mg total protein after 5 minutes for (mouse/surface bound) islet like cell clusters whose proportion of insulin secreting cells is 31.5 percent (Lumelsky et al., 2001. Science 292:1389-1394). Since, according to the authors of the aforementioned study, the average protein content of cells is 20 picograms, and since one milligram insulin corresponds to approximately 25.5 units of insulin, the maximal total insulin secretion capacity and insulin secretion rate of such clusters can be calculated to be 0.48 microunits per one hundred thousand insulin producing cells, and 5.7 microunits per one hundred thousand insulin producing cells per hour, respectively.

As described hereinabove, the method of the present invention can be used to generate cultured embryonic stem cell derived islet like clusters including insulin producing cells having an insulin secretion rate capacity of 955 microunits insulin per one hundred thousand cells per hour, and having a total insulin secretion capacity of 955 microunits insulin per one hundred thousand cells. Such an insulin secretion rate capacity and such a total insulin secretion capacity of the insulin producing cells of the present invention respectively represent about a 170-fold increase over the maximal insulin secretion rate capacity of 5.7 microunits insulin per one hundred thousand cells per hour, and about a 2000-fold increase over the maximal insulin secretion capacity of 0.48 microunits insulin per one hundred thousand cells of the insulin producing cells of prior art cultured embryonic stem cell derived islet like clusters (Lumelsky et al., 2001. Science 292:1389-1394).

Thus, the insulin producing cells of the present invention are vastly superior for producing insulin relative to the insulin producing cells of prior art cultured embryonic stem cell derived islet like clusters. It will be appreciated that such an optimal insulin secretion rate capacity and total insulin secretion capacity of the insulin producing cells of the present invention is clearly ideal for essentially any application requiring optimal insulin production.

As described hereinabove, various pharmacological agents can be used to upregulate or downregulate insulin secretion by the insulin producing cells of the present invention. Such pharmacological control may be advantageously employed for modulating insulin secretion by the insulin producing cells of the present invention in the context of various application.

Purification of Insuling Secreting Cells and Clusters

The capacity of the method of the present invention to generate islet like clusters under conditions suitable for inhibiting the growth of substantially non insulin producing cells, such as mesenchymal cells, is still another unique feature of the present invention relative to prior art methods of generating cultured embryonic stem cell derived islet like clusters. This capacity is advantageous, for example, for optimizing the proportion of islet cell types in clusters, and hence for generating optimally differentiated clusters. These advantages are ideal in applications requiring optimally differentiated pancreatic islets.

Differentiation of Insuling Secreting Cells and Clusters

The capacity of the method of the present invention to generate cell clusters including cells displaying the above listed the broad spectrum of characteristics associated with phenotypes of islet endocrine precursor cells, islet alpha cells, islet beta cells, islet delta cells, and islet neuronal cells indicates that such clusters include cells being highly differentiated along the alpha, beta, and delta cell lineages. Such cell types represent the great majority of normal pancreatic islet cells. Thus, by virtue of including such highly differentiated islet cell-like cells, the clusters including the insulin producing cells of the present invention are thereby capable of optimally performing the functions of normal pancreatic islets, and are hence also optimal for applications requiring optimally differentiated pancreatic islets.

For example, the inclusion of cells capable of producing islet cell specific hormones, such as somatostatin, glucagon, or insulin in the clusters is advantageous for applications in which islet cell specific cellular production of such hormones is desired. Furthermore, the inclusion of cells displaying an islet endocrine precursor cell phenotype in the clusters confers optimal differentiative plasticity and/or proliferation potential to such clusters.

Thus, the method of the present invention can be used to generate highly differentiated, highly functional human islet like cell clusters including cells capable of producing high levels of human insulin and being maintainable in culture for at least 14 days.

Method of Producing Insulin

According to another aspect of the present invention, there is provided a method of producing insulin.

The method is effected by generating the insulin producing cells of the present invention as described hereinabove, and preferably harvesting the insulin produced thereby.

In order to produce optimal levels of insulin in an optimally harvestable form, the cells are preferably cultured, according to the culturing protocol set forth in the Examples section below, so as to display an insulin secretion rate of 955 microunits insulin per one hundred thousand cells per hour and a total insulin secretion capacity of 955 microunits insulin per one hundred thousand cells.

Since, according to the present invention, the islet cell progenitors are cultured in a liquid medium to generate the cells of the present invention, and since the cells of the present invention secrete insulin in this medium, the secreted insulin may be conveniently and efficiently harvested by collecting such a medium. Such collection is preferably performed by centrifuging the cell culture, and collecting the resultant supernatant so as to easily and efficiently recover the secreted insulin in the absence of cellular or particulate contamination.

Following recovery thereof, the medium may constitute a suitable insulin preparation as such for various applications not requiring highly purified insulin. Alternately, for applications requiring highly purified insulin, the harvested medium may be advantageously subjected to a suitable chromatographic separation process so as to isolate the insulin to a desired degree of purity and/or yield. Ample guidance for chromatographically purifying insulin from a solution is provided in the literature of the art (for example, refer to: W. S. Hancock (ed.), “High Performance Liquid Chromatography in Biotechnology”, John Wiley & Sons, 1990).

Thus, the insulin producing cells of the present invention can be used to produce optimal quantities of easily purified human insulin which can be used, for example, to treat diabetes according to standard treatments, as described above.

Since, as described above, donor derived pancreatic islet cells, such as beta cells, can also be used to treat pancreatic diseases associated with islet pathogenesis, such as diabetes, and since, as described hereinabove, the insulin producing cells of the present invention constitute highly differentiated beta cell like cells capable of physiologically regulated high-level insulin secretion, and since the clusters including such cells constitute highly differentiated, highly functional pancreatic islet like cell clusters, such cells and clusters can be used to treat such diseases.

Method of Treating Pancreatic Disease

Therefore, according to the present invention, there is provided a method of treating a pancreatic disease in a subject.

The method is effected by administering a therapeutically effective dose of the insulin producing cells of the present invention to the subject.

Various methods of administering the insulin producing cells may be envisaged, including for example, via injection or catheter based delivery to the pancreas or liver of the subject, with or without surgical organ exposure. Preferably, the insulin producing cells are administered intra-hepatically via a transabdominal catheter, as described in further detail hereinbelow.

As used herein, a therapeutically effective dose is an amount sufficient to effect a beneficial or desired clinical result, which dose could be administered in one or more administrations. Preferably, a single administration is employed. The injection can be administered into various regions of the pancreas, depending on the type of pancreatic disease.

Since slight variations in insulin production and secretion can be significant in therapeutic uses, the level and/or rate of insulin produced, and optionally secreted, from such cells or clusters is preferably determined following generation thereof.

Various methods of determining the insulin producing/secreting capacity of such cells may be employed (for example, refer to: Lumelsky et al., 2001. Science 292:1389-1394). Preferably, the insulin secretion capacity of the insulin producing cells of the present invention is measured using a commercial microparticle enzyme immunoassay, as described in the Examples section below.

The method is preferably applied to treat diabetes in a human subject having such a disease. The method can also be applied to inhibit future onset or development of such a disease, so as to thereby inhibit such onset or development.

The present invention can be advantageously used to treat type II diabetes, or more preferably, type I diabetes.

As described hereinabove, the insulin producing cells of the present invention have the capacity to secrete, under physiological regulation, insulin at a broad range of secretion rates. Thus, the insulin producing cells of the present invention can be used, for example, to secrete physiologically regulated therapeutic levels of insulin so as to provide glycemic control when administered to the subject, thereby treating the disease in the subject.

Since, as described hereinabove, donor derived islet cells, such as beta cells, have been previously employed to treat pancreatic disease, such as diabetes, and since the insulin producing cells of the present invention are highly similar to such donor derived cells, it is well within the grasp of the ordinarily skilled artisan to determine a therapeutically effective dose for a human based on the guidance provided by the literature of the art. Preferably, determination of an effective dose can further be based on factors individual to each subject, including, for example, weight, age, physiological status, medical history, and parameters related to the pancreatic disease, such as, for example, the residual insulin secretion capacity of a diabetic subject. One skilled in the art, specifically a doctor, more preferably an endocrinologist, would be able to determine the number of insulin producing cells of the present invention that would constitute an effective dose, and the optimal mode of administration thereof without undue experimentation.

Ample guidelines for performing therapeutic transplantation of cell clusters including insulin secreting cells (i.e. pancreatic islets) are available in the literature of the art [for example, refer to guidelines provided by the National Institutes of Diabetes and Digestive and Kidney Diseases (NIDDK; http://www.niddk.nih.gov); Titus T. et al., 2000. Exp. Rev. Mol. Med. 6 September, http://www-ermm.cbcu.cam.ac.uk/00001861h.htm; and Norman D J. and Turka L A. (eds.), “Primer on Transplantation” 2nd ed. Mt Laurel, N.J., American Society of Transplantation, Maiden, Mass, Blackwell Science, 2001]

For administration of the insulin secreting cells, according to standard techniques, a surgeon uses ultrasound to guide placement of a small catheter through the upper abdomen and into the liver of the subject. The insulin secreting cells are then injected through the catheter into the liver. The patient preferably receives a local anesthetic, however if the subject cannot tolerate local anesthesia, the surgeon may use general anesthesia and perform the transplant through a small incision. Typically, for a 70 kilogram subject, a suitable transplant consists of about one million pancreatic islets. It takes some time for the administered cells to attach to new blood vessels and begin releasing insulin, hence following transplantation, the blood glucose levels of the subject are closely monitored and exogenous insulin is administered as needed until glycemic control is achieved [National Institutes of Diabetes and Digestive and Kidney Diseases (NIDDK; http:H/www.niddk.nih.gov)].

As described hereinabove, the insulin producing cells of the present invention may be derived from established stem cell lines, which are derived from cells which have never formed part of a tissue being further developed than the blastocyst stage and which would normally be allogeneic with the subject. Alternately, the insulin producing cells of the present invention may be derived from stem cells generated from differentiated cells, such as differentiated cells of a mammalian subject, in which case the insulin producing cells of the present invention would be syngeneic with the subject.

Thus, the method may be practiced by administering insulin producing cells of the present invention being syngeneic, or more preferably being allogeneic with the subject.

It will be recognized by the skilled practitioner that when administering allogeneic cells or tissues to a subject, there is routinely immune rejection of such cells or tissues by the subject. Thus, the method of the present invention preferably further comprises treating the subject with an immunosuppressive regimen, preferably prior to such administration, so as to inhibit such rejection. Immunosuppressive protocols for inhibiting allogeneic graft rejection, for example via administration of cyclosporin A, immunosuppressive antibodies, and the like are widespread and standard practice in the clinic.

Depending on the application and purpose, the insulin producing cells of the present invention may be advantageously administered to the subject at any point during any of the various culturing stages described hereinabove.

Preferably, in order to treat pancreatic diseases requiring high levels of insulin secretion, such as diabetes, the cells are administered after being subjected to the culturing conditions suitable for formation of surface bound clusters, more preferably after being subjected to the culturing conditions suitable for maintaining such cells in culture for 14 days, as described hereinabove, so as to generate cells having maximal insulin secretion capacity.

As described hereinabove, the optimal in-vitro longevity of the insulin producing cells of the present invention, and of the clusters including such cells, confers optimal flexibility for the timing of the use of the insulin producing cells of the present invention. This is particularly useful for such in-vivo cell therapy protocols which are characterized by stages of variable duration, such as, for example, attainment of optimal immunosuppression in the subject prior to allogeneic cell administration. Such optimal in-vitro longevity is also useful for generating optimal numbers of cells since it enables an optimal number of culture batches to be asynchronously produced while still being simultaneously harvestable.

Alternately, the insulin producing cells of the present invention may be advantageously administered after being subjected to the culturing conditions suitable for formation of surface bound clusters. As described hereinabove, following this culturing stage the capacity of carbachol to induce a decrease in insulin secretion by the insulin producing cells of the present invention while simultaneously increasing that of normal beta cells can be useful for controlling the balance of insulin secretion between the administered insulin producing cells of the present invention and the endogenous beta cells of the subject.

Depending on the application and purpose, the insulin producing cells of the present invention may be administered as a single cell suspension, optionally following isolation thereof, or preferably by administration of clusters containing such cells.

Such administration of islet like clusters containing the insulin producing cells of the present invention is optimal for treatment of diabetes, since, as described above, this disease been shown to be treatable by administration of donor derived islets. Islet transplantation is optimal for treating diabetes since islets provide the integrated organ structure, including islet neuronal cells responsible for optimal, synchronized, insulin secretion by beta cells. Since the clusters including the insulin producing cells of the present invention include a broad spectrum of differentiated cells, including cells displaying characteristics of a neuronal islet cell phenotype, such clusters are optimally suitable for providing the aforementioned integrated organ structure, and hence for use in treating diabetes. Furthermore, the inclusion of cells displaying an islet endocrine precursor cell phenotype in the clusters including the insulin producing cells of the present invention, as described hereinabove, confers upon such clusters the optimal differentiative plasticity and/or proliferative capacity required for optimal in-situ engraftment in the subject.

Critically, since essentially unlimited numbers of human pancreatic islet like clusters may be conveniently generated using the culturing method of the present invention, for example by culture scale-up of culture volumes and/or of numbers of cultures, the method of the present invention clearly overcomes the major obstacle to treatment of diabetes using donor derived islets which cannot practically be obtained in sufficient numbers.

Thus, in conclusion, the present invention provides methodology which can be used to generate cultured human islet like clusters having a higher proportion of insulin producing cells and a higher insulin secretion capacity than all prior art cultured human islet like clusters. In addition, the cultured islet like clusters of the present invention exhibit significantly greater in-vitro longevity than all prior art cultured islet like clusters.

As a result, the present invention greatly facilitates treatment of disorders associated with insulin deficiency.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-II Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Generation of High-Level Insulin Secreting, Highly Differentiated Human Pancreatic Islet Like Cell Clusters by In-Vitro Culture of Embryonic Stem Cells

Diabetes is a disease of tremendous medical and economic impact. One approach which has been proposed for treating diabetes involves administering functional pancreatic islets generated by in-vitro culture. However, all prior art approaches of generating such islets in-vitro have failed to provide islets being optimally differentiated, containing optimal proportions of insulin secreting cells, being capable of secreting optimal levels of insulin, and being optimal for human administration. While reducing the present invention to practice, the present inventors have uncovered a method of culturing of human embryonic stem cells to generate highly differentiated, human pancreatic islet like cell clusters containing cells capable of secreting high levels of insulin, as follows.

Materials and Methods:

Cell Culture:

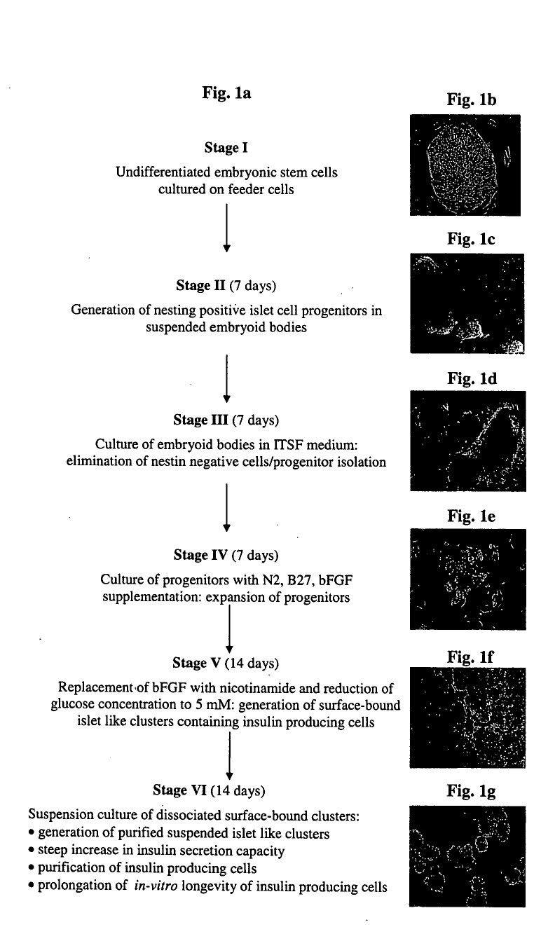

Undifferentiated embryonic stem cell lines H13 (passages 45-50; Thomson J A. et al., 1998. Science 282:1145), I6 (passages 45-50; Amit M, Itskovitz-Eldor J., 2000. J Anat. 200:225), and H9.2 (passages 45-80; Amit M. et al., 2000. Dev Biol. 227:271) which is a cloned line of H9 (Thomson J A. et al., 1998. Science 282:1145) were cultured so as to generate highly differentiated pancreatic islets and tissues was achieved the following novel culturing methodology (schematized in FIG. 1a).

Stage I—undifferentiated stem cell culture: Undifferentiated human embryonic stem cells were grown on mitotically inactivated mouse embryonic fibroblast (MEF) cells in 80 percent knockout DMEM, 20 percent knockout serum replacement, 1 millimolar glutamine and 1 percent non-essential amino acids solution, 0.1 millimolar 2-mercaptoethanol and 4 nanograms/ml bFGF.

Stage II—embryoid body formation: Undifferentiated human embryonic stem cells were transferred to non adherent plastic Petri dishes (Ein-Shemer) using 1 mg/ml type IV collagenase in order to allow their aggregation into embryoid bodies. The resultant embryoid bodies were cultured for 7 days in 80 percent knockout DMEM/20 percent defined FBS medium supplemented with 1 millimolar glutamine and 1 percent non-essential amino acids solution.

Stage III—generation of pancreatic progenitors: The 7 day old embryoid bodies were plated at a density of 300 embryoid bodies per well in 6 well, adherent cell culture coated culture flasks (Nunc) and grown for an additional 7 days in “Medium 1” (DMEM/F12 1:1 supplemented with 10 milligrams/liter insulin, 6.7 milligrams/liter sodium selenite, 5.5 milligrams/liter transferrin, 1 millimolar glutamine, and 5 micrograms/ml fibronectin). This stage produces a highly enriched population of nestin positive pancreatic progenitor cells.

Stage IV—expansion of pancreatic progenitors: Embryoid bodies from Stage III culture were dissociated to single cells by treatment with PBS containing 0.5 millimolar EDTA, 1 percent FBS, and 0.1 millimolar 2-mercaptoethanol. The dissociated embryoid body cells were transferred to adherent tissue culture plates (precoated with either 0.1 percent gelatin or 20 nanograms/ml poly L-lysine) at a concentration of 2×10⁷ cells/ml in “Medium 2” (DMEM/F12 1:1 supplemented with N2 and B27 DMEM supplements according to the manufacturer's instructions, 1 millimolar glutamine, and 10 nanograms/ml bFGF) and cultured for 7 days with a medium change every 2 days. This stage served to expand pancreatic progenitor cells generated during Stage III culture.

Stage V—generation of surface bound, insulin producing, islet like cell clusters: Cells were transferred from the bFGF supplemented Medium 2 to “Medium 3” (glucose-free DMEM supplemented with 10 millimolar nicotinamide) and cultured for 7 days. Since glucose free DMEM was used in Medium 3, the glucose concentration therein was reduced from 17.5 millimolar in Medium 2 to 5 millimolar in Medium 3.

Stage VI—generation of isolated, highly differentiated, high-level insulin producing suspended islet like cell clusters having long term in-vitro longevity: Surface bound islet like cell clusters from Stage V culture were dissociated by trypsin-EDTA treatment and cultured for up to 2 weeks under non adherent “suspension” conditions in uncoated plastic Petri dishes (Ein-Shemer) in Medium 3. This step resulted in prolongation of islet like cell cluster survival beyond 2 weeks, a sharp increase in insulin secretion, and a reduction in the proportion of neuronal and mesenchymal cells in the cell clusters. The use of such suspension culturing conditions has been previously demonstrated to prolong the longevity of insulin secreting human islets derived from adult donor derived cells (Zhao M. et al., 2002. Transplantation 73: 1454-1460).

Tissue culture media and supplements: Knockout Dulbecco's modified Eagle's medium (DMEM), Knockout Serum Replacement, glutamine, non-essential amino acid solution, 2-mercaptoethanol, basic fibroblast growth factor (bFGF), type IV collagenase, 100× Insulin-Transferrin-Selenium solution (Cat. no. 41400-045, Gibco-BRL), and N2 and B27 DMEM supplements were obtained from Gibco-BRL; defined fetal bovine serum (FBS) was obtained from Hyclone, Logan, Utah; fibronectin was obtained from Roche; and poly L-lysine and nicotinamide were obtained from Sigma.

RT-PCR analysis of pancreas specific gene expression: Total RNA was isolated from differentiated human embryonic stem cells using Tri-Reagent (Sigma), according to the manufacturer's recommended protocol. cDNA was synthesized from 1 microgram total RNA using MMLV reverse transcriptase RNase H minus (Promega, Madison, Wis., USA). The mRNA species amplified, and the PCR primers and reaction conditions used to amplify such species are shown in Table 1. PCR amplification products were size fractionated and visualized via 2 percent agarose gel

TABLE 1

PCR primer pairs used for RT-PCR* amplification
of mRNA.

| | | | |
|---|---|---|---|
| | | Anneal- | |
| | | ing | PCR |
| | | temp- | pro- |
| era- | duct | | |
| mRNA | | ture | size |
| species | Upstream/Downstream primers | (° C.) | (bp) |

| | | | |
|---|---|---|---|
| glu- | 5′-AGGCAGACCCACTCAGTGAT-3′ | 55 | 308 |
| cagon | (SEQ ID NO: 1)/ | | |
| | 5′-AACAATGGCGACCTCTTCTG-3′ | | |
| | (SEQ ID NO: 2) | | |
| Pax6 | 5′-CCGAGAGTAGCGACTCCAG-3′ | 65 | 239 |
| | (SEQ ID NO: 3)/ | | |
| | 5′-CTTCCGGTCTGCCCGTTC-3′ | | |
| | (SEQ ID NO: 4) | | |
| gluco- | 5′-AAGAAGGTGATGAGACGGATGC-3′ | 68 | 230 |
| kinase | (SEQ ID NO: 5)/ | | |
| (GCK) | 5′-CATCTGGTGTTTGGTCTTCACG-3′ | | |
| | (SEQ ID NO: 6) | | |
| Beta2 | 5′-GCTCGAAGCCATGAACGCAG-3′ | 55- | 583 |
| | (SEQ ID NO: 7)/ | 65 | |
| | 5′-GCTGTCCATGGTACCGTAAG-3′ | | |
| | (SEQ ID NO: 8) | | |
| Nkx6.1 | 5′-GTTCCTCCTCCTCCTCTTCCTC-3′ | 55 | 381 |
| | (SEQ ID NO: 9)/ | | |
| | 5′-AAGATCTGCTGTCCGGAAAAAG-3′ | | |
| | (SEQ ID NO: 10) | | |
| Glut2 | 5′-AGGACTTCTGTGGACCTTATGTG-3′ | 55 | 231 |
| | (SEQ ID NO: 11)/ | | |
| | 5′-GTTCATGTCAAAAAGCAGGG-3′ | | |
| | (SEQ ID NO: 12) | | |
| Isl1 | 5′-GATTTCCCTATGTGTTGGTTGC-3′ | 60 | 827 |
| | (SEQ ID NO: 13)/ | | |
| | 5′-CTTCCACTGGGTTAGCCTGTAA-3′ | | |
| | (SEQ ID NO: 14) | | |
| Pax4 | 5′-GTGGGCAGTATCCTGATTCAGT-3′ | 55 | 308 |
| | (SEQ ID NO: 15)/ | | |
| | 5′-TGTCACTCAGACACCTTTCTGG-3′ | | |
| | (SEQ ID NO: 16) | | |
| in- | 5′-AGCCTTTGTGAACCAACACC-3′ | 55 | 245 |
| sulin | (SEQ ID NO: 17)/ | | |
| | 5′-GCTGGTAGAGGGAGCAGATG-3′ | | |
| | (SEQ ID NO: 18) | | |
| Pdx1 | 5′-GGATGAAGTCTACCAAAGCTCACGC-3′ | 65 | 230 |
| | (SEQ ID NO: 19)/ | | |
| | 5′-CCAGATCTTGATGTGTCTCTCGGTC-3′ | | |
| | (SEQ ID NO: 20) | | |
| so- | 5′-GTACTTCTTGGCAGAGCTGCTG-3′ | 55 | 2179 |
| mato- | (SEQ ID NO: 21)/ | | |
| statin | 5′-CAGAAGAAATTCTTGCAGCCAG-3′ | | |
| | (SEQ ID NO: 22) | | |
| Ngn3 | 5′-CAATCGAATGCACAACCTCA-3′ | 55 | 2179 |
| | (SEQ ID NO: 23)/ | | |
| | 5′-GGGAGACTGGGGAGTAGAGG-3′ | | |
| | (SEQ ID NO: 24) | | |
| GAPDH | 5′-AGCCACATCGCTCAGACACC-3′ | 55 | 2179 |
| | (SEQ ID NO: 25)/ | | |
| | 5′-GTACTCAGCGGCCAGCATCG-3′ | | |
| | (SEQ ID NO: 26) | | |

*40 thermal cycles were employed for all amplifications except for Glut2 (35 cycles) and GAPDH (30 cycles)

Insulin secretion assay: Islet like cell clusters were washed twice in KRBH buffer (120 millimolar NaCl, 5 millimolar KCl, 2.5 millimolar CaCl₂, 1.1 millimolar NaHCO₃, 0.5 percent BSA, and 10 millimolar HEPES) and pre-incubated for 2 hours with KRBH buffer supplemented with 3.3 millimolar glucose. The cell clusters were then incubated for 1 hour in KRBH buffer supplemented with either 3.3 millimolar glucose (“low glucose”) or 16.7 millimolar glucose (“high glucose”). Insulin secretion levels were measured using a microparticle enzyme immunoassay (AXSYM system Insulin kit code B2DO10, Abbott Laboratories) which detects human insulin without cross reactivity to pro-insulin or C-peptide. Insulin secretion per cell number was determined by counting cells via haemocytometer. Assays were performed in a total volume of 250 microliters.

To determine if the cell clusters use physiological signaling pathways to regulate insulin release, cell clusters were incubated in the presence of various pharmacological agonists and antagonists of insulin secretion, and resultant insulin secretion levels were analyzed. electrophoresis.

Agonists: tolbutamide, a sulfonylurea inhibitor of ATP dependent potassium channel was added at a concentration of 10 micromolar; 3-isobutyl-1-methylxanthine (IBMX), an inhibitor of cyclic AMP, was added at a concentration of 100 micromolar; and carbachol, an agonist of muscarinic cholinergic receptors, was added at a concentration of 100 micromolar.

Antagonists: diazoxide, an activator of ATP dependent potassium channel, was added at a concentration of 500 micromolar; and nifedipine, a blocker of L-type calcium channel, was added at a concentration of 50 micromolar.

Immunohistochemical analysis of pancreatic hormone expression: Single cells from the end of Stage III, were seeded on 13 mm glass cover slides, in 6 well culture plates and grown under conditions of Stage IV-V culture. Cells were fixed for 20 minutes in PBS containing 4 percent paraformaldehyde. Cells were stained for insulin, glucagons or somatostatin, using LSAB+ Peroxidase Staining Kit (Dako), according to the manufacturer's instructions.

Immunofluorescence analysis of pancreatic hormone expression: Single cells were grown and fixed as described above. Cells were permeabilized using PBS containing 0.5 percent Triton X-100/1 percent rabbit serum, and incubated for 2 hours at room temperature with either guinea pig anti insulin or rabbit anti glucagon (DAKO) primary antibody. After being washed with PBS containing 0.5 percent Triton X-100/1 percent serum, FITC conjugated anti rabbit IgG secondary antibody (Sigma) was added to both rabbit and guinea pig antibody stained samples, which were then incubated overnight at 4 degrees centigrade. Alternately, mouse anti insulin (Sigma) primary antibody and FITC conjugated anti mouse IgG secondary antibody (Chemicon) were used to detect insulin. Cells labeled with secondary antibody were washed and mounted with mounting medium (Vectashield, Vector Lab, Calif.).

Stage VI clusters were seeded on 13 mm glass cover slides in 6-well culture plates. Forty-eight hours after seeding, cells were fixed for 20 minutes in 4% paraformaldehyde in PBS, permeabilized using 0.5% TritonX-100 in PBS/1% serum, and incubated overnight with the primary antibody mouse anti-insulin antibody (1:100 dilution; Sigma Chemicals Inc) with either rabbit anti-C-peptide (1:100 dilution; Linco Research Inc, St. Charles, Mich., USA), rabbit anti-glucagon (1:100 dilution) or rabbit anti-somatostatin antibody (1:200 dilution) (both from DAKO Corporation, Carpinteria, Calif., USA).

After rinsing, secondary anti-rabbit IgG FITC conjugated antibody (1:100 dilution) and anti-mouse IgG Cy3 conjugated antibody (1:100 dilution) (both from Sigma Chemicals Inc) were added to the samples, which were then incubated for an additional hour. Finally, the cells were rinsed once more and mounted with mounting media (VECTASHIELD, Vector Lab, Calif.). The slides were analyzed using a confocal microscope (Bio-Rad MRC 1024, Richmond, Calif.).

BrdU labeling: In order to determine the percent of the proliferating cells, the BrdU Streptvidin-Biotin labeling kit (Zymed, San-Francisco, Calif., USA) was used. The cells were incubated overnight with the BrdU labeling reagent (diluted 1:100). Following the incubation, cells were rinsed 2x PBS, fixed with 75% alcohol (20 minutes at room temperature), and then stained according to the manufacturer's recommended protocol.

TUNEL assay: A TUNEL assay was preformed in order to detect the amount of apoptotic cells within the clusters. Cells on glass coverslips were fixed for 20 minutes in 4% paraformaldehyde in PBS and then washed three times in PBS. Staining was preformed using the In Situ Cell Death Detection Kit (Roche, Mannheim, Germany) according to the manufacturer's recommended protocol. The reaction was stopped using 2×SSC buffer, washed, and was then subjected to immunofluoresence staining with mouse anti-insulin antibody (Sigma Chemicals Inc).

Experimental Results:

The appearance of the cells and tissues generated during Stages I-VI of culture are shown in FIGS. 1b-g, respectively. All results shown below were repeatable using cell lines H13, 16 and H9.2.

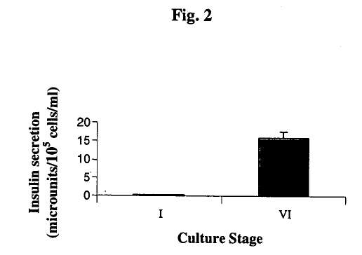

As shown in FIG. 2, insulin secretion was vastly enhanced in Stage VI suspended islet like cell clusters compared to Stage I undifferentiated embryonic stem cells, therefore the protocol employed can be used to generate insulin secreting islet like cell clusters from undifferentiated human embryonic stem cells.

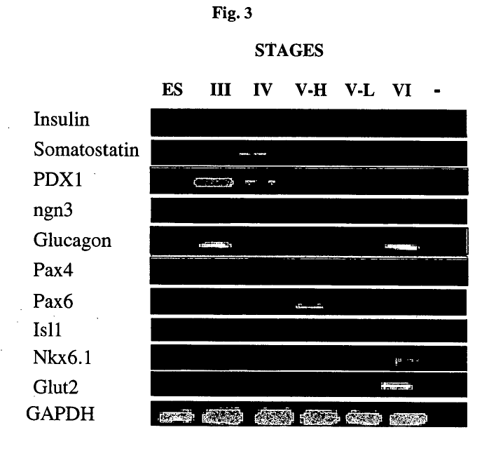

In order to analyze the level of pancreatic differentiation achieved using the culturing protocol of the present invention, cultures were analyzed for expression of pancreas specific genes. As shown in FIG. 3, there was an enhanced expression of pancreas genes in the differentiating hES cells. The transcription factor pancreatic duodenal homeobox 1 (PDX1) appeared mainly in stage III mRNA and decreased in stage IV. Glucagon and Neurogenine 3 (Ngn3) were highly expressed in stage III and then in stage VI. Somatostatin expression increased at stage IV in comparison to stage III and then disappeared. Pax4 expression was increased from stage III to IV and then decreased in stage V-H (high glucose in the medium), but when the glucose concentration was reduced, Pax4 was further increased (stage V-L, low glucose in the medium). Pax6 was noticed in all stages of differentiation. Insulin and other pancreatic beta cell specific genes such as Nkx6.1, Isl1, Glut2 and insulin were only noticed in mRNA from stage VI cells.

Thus, Stage VI cells display a highly differentiated islet of Langerhans beta cell phenotype.

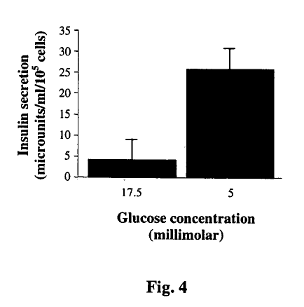

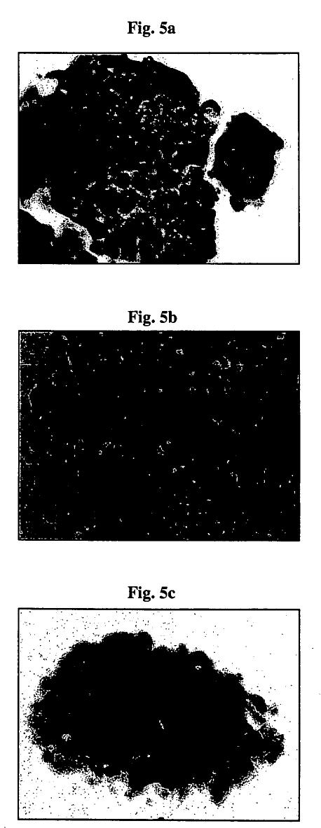

In order to analyze the specific contribution of the decrease in glucose concentration from 17.5 millimolar in Medium 2 used in Stage IV relative to the glucose concentration of 5 millimolar in Medium 3 used in Stage V to changes in insulin secretion by Stage V surface bound islet like cell clusters compared to Stage IV pancreatic progenitor cells, Stage V surface bound islet like cell clusters were either grown in Medium 3 (5 millimolar glucose, standard Stage V conditions) or Medium 3 modified to have a glucose concentration increased to 17.5 millimolar, and assayed for insulin secretion. Reduction of the glucose concentration from 17.5 millimolar to 5 millimolar in Medium 3 was shown to lead to sharply increased insulin secretion (FIG. 4). Thus, the decrease in glucose concentration in Medium 3 relative to Medium 2 contributed to the observed increase in insulin secretion levels. Insulin secretion levels in Stage V surface bound islet like cell clusters were found to display expression of insulin, glucagon, and somatostatin via immunohistochemical analysis (FIGS. 5a-c, respectively). In particular, such analysis revealed that a very high proportion, 60 to 80 percent, of the cells expressed insulin. Within cell clusters insulin positive stained cells were observed to be surrounded by other cell types, mainly neurons, and glucagon/somatostatin producing cells, and outside of the cell clusters mainly neurons and mesenchymal cells, were observed (FIG. 1f).

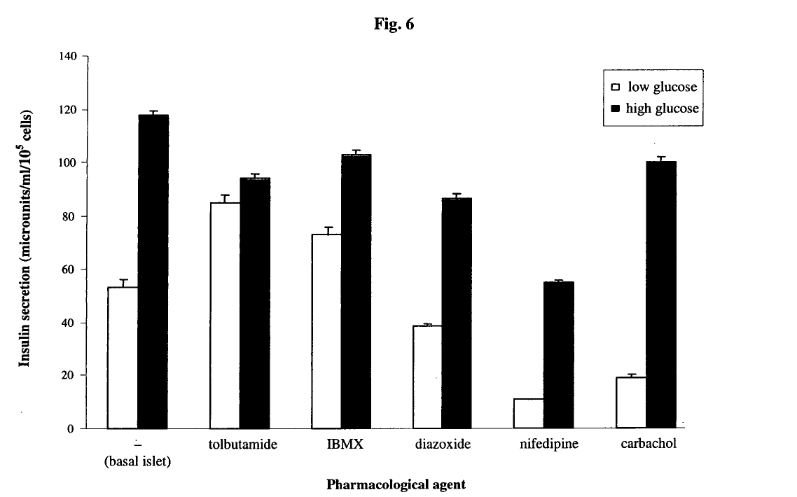

To determine whether islet like cell clusters employ beta cell specific physiological signaling pathways to regulate their insulin secretion, the effects of several agonists and antagonists on insulin secretion by Stage V surface bound islet like cell clusters were examined. As shown in FIG. 6, the observed insulin secretion profiles following treatment with “low glucose” (3.3 millimolar) and either the agonists tolbutamide (10 micromolar) or IBMX (100 micromolar), or the antagonists nifedipine (50 micromolar) or diazoxide (500 micromolar) were characteristic of pancreatic beta cells. Such results therefore provide further convincing evidence for differentiation of cultured cells along the pancreatic beta cell lineage.

Growth of the cells under Stage V culture conditions for a time period longer than 2 weeks resulted in take-over of the culture by non beta cell lineage cell types, such as neurons and fibroblasts, and disappearance of surface bound islet like cell clusters (data not shown).

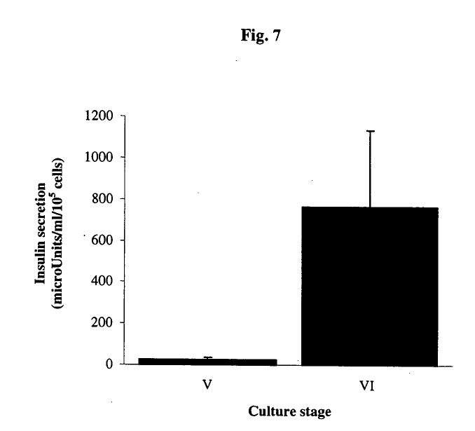

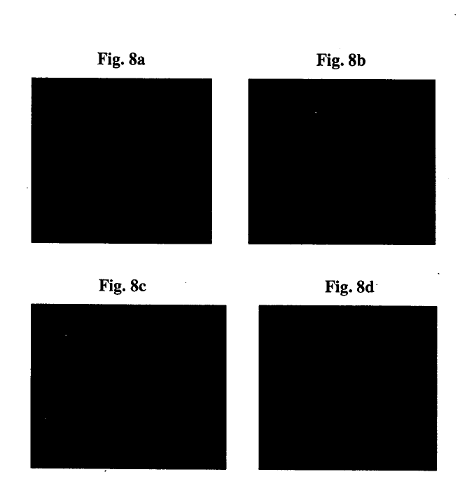

In order to prolong islet like cell cluster survival, surface bound cell clusters from Stage V culture were dissociated by trypsin-EDTA treatment and the dissociated cells were grown in suspension in Medium 3 on a non adherent substrate (Stage VI). Suspended islet like cell clusters were unexpectedly found to be formed by this procedure (FIG. 1f), and only cells that were aggregated into such cell clusters were observed to survive. Also unexpectedly, levels of insulin secretion were found to be sharply increased to 764 microunits per ml per hundred thousand cells in Stage VI suspended islet like cell clusters from 40 microunits per ml per hundred thousand cells to Stage V surface bound islet like cell clusters, as shown in FIG. 7. Similarly, the percentage of insulin producing cells was also unexpectedly found to be increased in Stage VI suspended islet like cell clusters relative to Stage V surface bound islet like cell clusters, as shown via immunohistochemical analysis (FIG. 5a). Examination via immunofluorescent confocal microscopy revealed that the percentage of insulin expressing cells in Stage VI suspended islet like cell clusters (FIGS. 8a-d) was 80 percent, the highest proportion of insulin expressing cells ever achieved in a cell culture derived from embryonic stem cells.

Thus, the non adherent conditions used in Stage VI culture lead to increased levels of insulin secretion and an increased proportion of insulin secreting cells relative to adherent conditions used in Stage V culture.

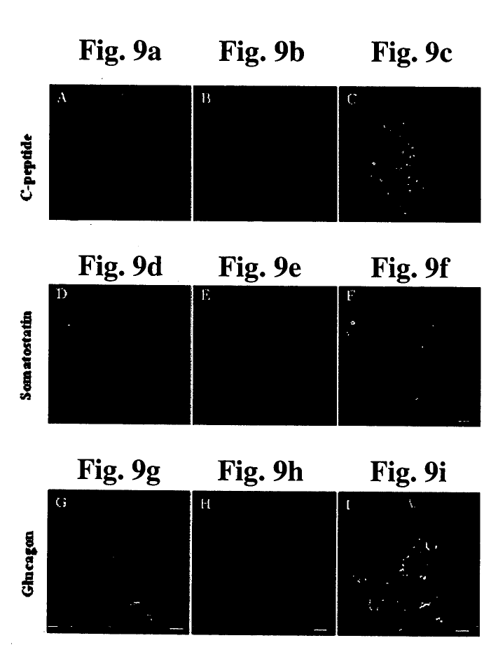

Forming clusters were examined by immunofluoresence for the expression of insulin, C-peptide, somatostatin and glucagon (FIGS. 9a-i). The results revealed a high percentage of insulin-expressing cells in the clusters. The cells co-expressed both insulin and C-peptide, indicating an indubitable production of insulin. In addition to insulin, most cells also co-expressed glucagon and somatostatin. This is a well-known developmental phenomenon of human and mouse embryonic pancreas (Polak et al., 2000. Diabetes 49:225-32; Teitelman G. et al., 1993. Development 118:1031-9; Chiang M K, Melton D A, 2003. Dev Cell. 4:383).

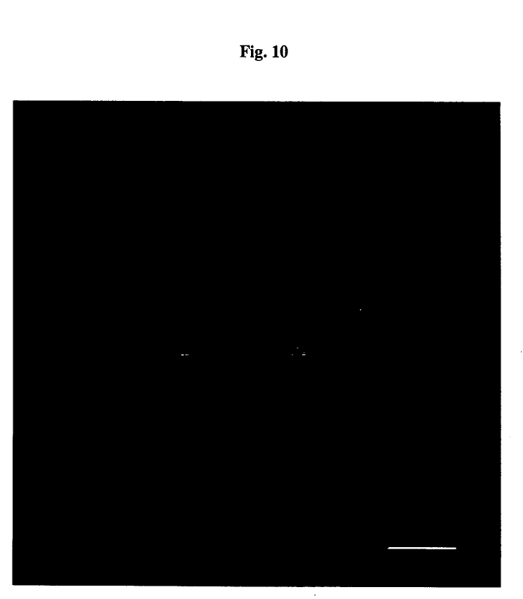

Cell proliferation was further tested using a BrdU staining kit (Zymed, San Francisco, Calif.). BrdU was incorporated into proliferating cells at the S-phase. Many of the cells at stage VI were stained with BrdU and thus confirmed the clusters' proliferation (data not shown). Furthermore, the aggregation step increased the survivability of the cells, from one week at stage V to over a month at stage VI. The cells were further tested with TUNEL combined with insulin staining. Some of the cells stained positively for both TUNEL and insulin, suggesting apoptosis. However, in contrast to previous studies (Rajagopal J. et al., 2003. Science 299:363), most of the cells from stage VI that stained positive for insulin were not apoptotic (FIG. 10). Both BrdU staining, TUNEL and C-peptide staining of the cells confirmed that stage VI cells still-proliferate and produce de novo insulin.

Conclusion: The above described results demonstrate for the first time generation of highly functional, highly differentiated beta cells and pancreatic islet like cell clusters by in-vitro culture of human embryonic stem cells. Furthermore, the above described results demonstrate that the present method can be used to generate cultures containing far higher percentages of insulin expressing cells and having a significantly higher insulin production capacity, and having longer in-vitro longevity than all prior art cultures. Importantly, the presently described results demonstrate that the present invention can be used to generate cultured pancreatic islet like cell clusters composed of proliferating, non-apoptotic insulin-producing cells after at least one month in culture.

Thus, due to the aforementioned characteristics, the method of the present invention can be used to generate insulin secreting human cells and tissues being optimal for treating diseases associated with insulin deficiency such as Type I diabetes in humans, as well as, for example, to generate in-vitro cultures of human pancreatic beta cells and islet of Langerhans like tissues being optimal for screening and testing of pancreatic beta cell specific drugs, for industrial production of human pancreatic hormones such as insulin, glucagon, and somatostatin, and for in-vitro study of islet cell and tissue biology.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

1-192. (canceled)

193. A method of generating cells capable of secreting insulin, the method comprising:
(a) subjecting mammalian embryonic stem cells to a first set of culturing conditions selected suitable for differentiation of at least a portion of said mammalian embryonic stem cells into cells displaying at least one characteristic associated with a pancreatic islet cell progenitor phenotype; and
(b) subjecting said cells displaying at least one characteristic associated with a pancreatic islet cell progenitor phenotype to a second set of culturing conditions selected suitable for formation of surface bound cell clusters including insulin producing cells, thereby generating cells capable of secreting insulin.

194. A method of producing insulin, the method comprising:
(a) subjecting mammalian embryonic stem cells to a first set of culturing conditions selected suitable for differentiation of at least a portion of said mammalian embryonic stem cells into cells displaying at least one characteristic associated with a pancreatic islet cell progenitor phenotype; and
(b) subjecting said cells displaying at least one characteristic associated with a pancreatic islet cell progenitor phenotype to a second set of culturing conditions selected suitable for formation of surface bound cell clusters including insulin producing cells, thereby producing the insulin.

195. The method of claim 193, further comprising:
(c) isolating said surface bound cell clusters and optionally isolating said insulin producing cells therefrom.

196. The method of claim 193, further comprising:
(c) dissociating said surface bound cell clusters into single cells including said insulin producing cells; and
(d) subjecting said single cells to a third set of culturing conditions selected suitable for maintaining said insulin producing cells in culture for at least 14 days.

197. The method of claim 196, further comprising:
(e) isolating said insulin producing cells.

198. The method of claim 196, wherein said third set of culturing conditions is selected suitable for maintaining said insulin producing cells in suspended cell clusters.

199. The method of claim 198, wherein said suspended cell clusters are characterized by a proportion of said insulin producing cells of at least 4 percent.

200. The method of claim 198, wherein an insulin secretion rate capacity of said insulin producing cells of said suspended cell clusters is at least 6 microunits insulin per one hundred thousand cells per hour.

201. The method of claim 194, further comprising:
(c) harvesting the insulin.

202. The method of claim 198, further comprising:
(e) isolating said suspended cell clusters.

203. The method of claim 196, wherein said third set of culturing conditions is selected suitable for inhibiting growth of substantially non insulin producing cells.

204. The method of claim 203, wherein said substantially non insulin producing cells are neurons and/or mesenchymal cells.

205. The method of claim 196, wherein said dissociating said surface bound cell clusters into single cells is effected by trypsinization of said surface bound cell clusters.

206. The method of claim 196, wherein said third set of culturing conditions includes a condition selected from the group consisting of a substantially serum free culture medium, a basic fibroblast growth factor free culture medium, a culture medium including nicotinamide, a culture medium including a synthetic serum supplement, a culture medium including glucose at a concentration of 15 millimolar or less, and inhibiting adherence of said insulin producing cells to a surface.

207. The method of claim 193, wherein said first set of culturing conditions is selected suitable for inducing formation of embryoid bodies.

208. The method of claim 193, wherein said first set of culturing conditions is selected capable of inhibiting adherence of said mammalian embryonic stem cells to a surface.

209. The method of claim 193, wherein said at least one characteristic associated with a pancreatic islet cell progenitor phenotype is expression and optionally display of nestin.

210. The method of claim 193, further comprising:
(c) dissociating said cells displaying at least one characteristic associated with a pancreatic islet phenotype into single cells displaying at least one characteristic associated with a pancreatic islet phenotype; and
(d) subjecting said single cells displaying at least one characteristic associated with a pancreatic islet cell progenitor phenotype to a fifth set of culturing conditions selected suitable for proliferation of said cells displaying at least one characteristic associated with a pancreatic islet cell progenitor phenotype prior to step (b).

211. The method of claim 210, wherein said fourth set of culturing conditions includes a culturing condition selected from the group consisting of a substantially serum free culture medium, a culture medium including insulin, a culture medium including transferrin, a culture medium including fibronectin, a culture medium substantially including selenium, and facilitating adherence of said cells displaying at least one characteristic associated with a pancreatic islet cell progenitor phenotype to a surface.

212. The method of claim 193, wherein said second set of culturing conditions is selected suitable for formation of cell clusters including cells displaying at least one characteristic associated with a pancreatic islet cell phenotype selected from the group consisting of an endocrine cell precursor phenotype, an alpha cell phenotype, a beta cell phenotype, a delta cell phenotype, and a neuronal cell phenotype.

213. The method of claim 193, wherein said second set of culturing conditions is selected suitable for formation of cell clusters including insulin producing cells capable of displaying a change in an insulin secretion in response to a drug selected from the group consisting of an increase in said insulin secretion wherein said drug is tolbutamide, an increase in said insulin secretion wherein said drug is IBMX, a decrease in said insulin secretion wherein said drug is diazoxide, a decrease in said insulin secretion wherein said drug is nifedipine, and a decrease in said insulin secretion wherein said drug is carbachol.

214. The method of claim 193, wherein said mammalian embryonic stem cells are human embryonic stem cells.

215. The method of claim 214, wherein said human embryonic stem cells are selected from the group consisting of I6 cells, H9 cell derived cells, and H13 cells.

216. The method of claim 215, wherein said H9 cell derived cells are H9.2 cells.

217. An insulin producing cell cluster comprising insulin producing cells being maintainable in culture for at least 14 days, wherein a proportion of said insulin producing cells in the cell cluster is at least 4 percent.

218. The insulin producing cell cluster of claim 217, wherein said proportion of said insulin producing cells in the cell cluster is at least 32 percent.

219. The insulin producing cell cluster of claim 217, wherein an insulin secretion rate capacity of said insulin producing cells is at least 6 microunits insulin per one hundred thousand cells per hour.

220. The insulin producing cell cluster of claim 217, wherein the cell cluster further comprises cells displaying at least one characteristic associated with a pancreatic islet cell phenotype selected from the group consisting of an endocrine cell precursor phenotype, an alpha cell phenotype, a beta cell phenotype, a delta cell phenotype, and a neuronal cell phenotype

221. The insulin producing cell cluster of claim 217, wherein said insulin producing cell cluster produces human insulin.

222. The insulin producing cell cluster of claim 217, wherein said insulin producing cell cluster includes human cells.

223. The insulin producing cell cluster of claim 222, wherein said human cells have a genotype of I6 cells, H9 cell derived cells, and H13 cells.

224. The insulin producing cell cluster of claim 223, wherein said H9 cell derived cells are H9.2 cells.

225. A method of treating a pancreatic disease in a subject, the method comprising:
(a) subjecting mammalian embryonic stem cells to a first set of culturing conditions selected suitable for differentiation of at least a portion of said mammalian embryonic stem cells into cells displaying at least one characteristic associated with a pancreatic islet cell progenitor phenotype;
(b) subjecting said cells displaying at least one characteristic associated with a pancreatic islet cell progenitor phenotype to a second set of culturing conditions selected suitable for formation of surface bound cell clusters including insulin producing cells; and
(c) administering a therapeutically effective dose of said insulin producing cells to the subject, thereby treating the pancreatic disease.

226. The method of claim 225, further comprising isolating said surface bound cell clusters and optionally said insulin producing cells therefrom prior to step (c).

227. The method of claim 225, further comprising:
(d) dissociating said surface bound cell clusters into single cells including said insulin producing cells; and
(e) subjecting said single cells to a third set of culturing conditions selected suitable for maintaining said insulin producing cells in culture for at least 14 days prior to step (c).

228. The method of claim 225, wherein a total insulin secretion capacity of said insulin producing cells of said suspended cell clusters is at least 0.50 microunits insulin per one hundred thousand cells.

229. The method of claim 225, wherein said mammalian embryonic stem cells are human embryonic stem cells.

230. The method of claim 229, wherein said human embryonic stem cells are selected from the group consisting of I6 cells, H9 cell derived cells, and H 13 cells.

231. The method of claim 230, wherein said H9 cell derived cells are H9.2 cells.

232. The method of claim 225, wherein said insulin producing cells are syngeneic with or allogeneic with the subject.

233. The method of claim 225, wherein the subject is a human or a non human mammal.

234. The method of claim 225, wherein said administering is effected by transplantation or injection of said insulin producing cells into the pancreas of the subject.


SEQUENCE LISTING (26 sequences)

SEQ ID NO: 1 - DNA, 20 bp, Artificial sequence
  Single strand DNA oligonucleotide
aggcagacccactcagtgat

SEQ ID NO: 2 - DNA, 20 bp, Artificial sequence
  Single strand DNA oligonucleotide
aacaatggcgacctcttctg

SEQ ID NO: 3 - DNA, 19 bp, Artificial sequence
  Single strand DNA oligonucleotide
ccgagagtagcgactccag

SEQ ID NO: 4 - DNA, 18 bp, Artificial sequence
  Single strand DNA oligonucleotide
cttccggtctgcccgttc

SEQ ID NO: 5 - DNA, 22 bp, Artificial sequence
  Single strand DNA oligonucleotide
aagaaggtgatgagacggatgc

SEQ ID NO: 6 - DNA, 22 bp, Artificial sequence
  Single strand DNA oligonucleotide
catctggtgtttggtcttcacg

SEQ ID NO: 7 - DNA, 20 bp, Artificial sequence
  Single strand DNA oligonucleotide
cctcgaagccatgaacgcag

SEQ ID NO: 8 - DNA, 20 bp, Artificial sequence
  Single strand DNA oligonucleotide
gctgtccatggtaccgtaag

SEQ ID NO: 9 - DNA, 22 bp, Artificial sequence
  Single strand DNA oligonucleotide
gttcctcctcctcctcttcctc

SEQ ID NO: 10 - DNA, 22 bp, Artificial sequence
  Single strand DNA oligonucleotide
aagatctgctgtccggaaaaag

SEQ ID NO: 11 - DNA, 23 bp, Artificial sequence
  Single strand DNA oligonucleotide
aggacttctgtggaccttatgtg

SEQ ID NO: 12 - DNA, 20 bp, Artificial sequence
  Single strand DNA oligonucleotide
gttcatgtcaaaaagcaggg

SEQ ID NO: 13 - DNA, 22 bp, Artificial sequence
  Single strand DNA oligonucleotide
gatttccctatgtgttggttgc

SEQ ID NO: 14 - DNA, 22 bp, Artificial sequence
  Single strand DNA oligonucleotide
cttccactgggttagcctgtaa

SEQ ID NO: 15 - DNA, 22 bp, Artificial sequence
  Single strand DNA oligonucleotide
gtgggcagtatcctgattcagt

SEQ ID NO: 16 - DNA, 22 bp, Artificial sequence
  Single strand DNA oligonucleotide
tgtcactcagacacctttctgg

SEQ ID NO: 17 - DNA, 20 bp, Artificial sequence
  Single strand DNA oligonucleotide
agcctttgtgaaccaacacc

SEQ ID NO: 18 - DNA, 20 bp, Artificial sequence
  Single strand DNA oligonucleotide
gctggtagagggagcagatg

SEQ ID NO: 19 - DNA, 25 bp, Artificial sequence
  Single strand DNA oligonucleotide
ggatgaagtctaccaaagctcacgc

SEQ ID NO: 20 - DNA, 25 bp, Artificial sequence
  Single strand DNA oligonucleotide
ccagatcttgatgtgtctctcggtc

SEQ ID NO: 21 - DNA, 22 bp, Artificial sequence
  Single strand DNA oligonucleotide
gtacttcttggcagagctgctg

SEQ ID NO: 22 - DNA, 22 bp, Artificial sequence
  Single strand DNA oligonucleotide
cagaagaaattcttgcagccag

SEQ ID NO: 23 - DNA, 20 bp, Artificial sequence
  Single strand DNA oligonucleotide
caatcgaatgcacaacctca

SEQ ID NO: 24 - DNA, 20 bp, Artificial sequence
  Single strand DNA oligonucleotide
gggagactggggagtagagg

SEQ ID NO: 25 - DNA, 20 bp, Artificial sequence
  Single strand DNA oligonucleotide
agccacatcgctcagacacc

SEQ ID NO: 26 - DNA, 20 bp, Artificial sequence
  Single strand DNA oligonucleotide
gtactcagcggccagcatcg